(19) United States
(12) Patent Application Publication
(10) Pub. No.: US 2023/0348982 A1
(43) Pub. Date: Nov. 2, 2023

(54) METHODS OF IDENTIFYING MARKERS OF GRAFT REJECTION

(71) Applicant: SIRONA GENOMICS, INC., Mountain View, CA (US)

(72) Inventors: Sujatha KRISHNAKUMAR, Mountain View, CA (US); Ming LI, Mountain View, CA (US); Peggy PALSGAARD, Mountain View, CA (US); Michael N. MINDRINOS, Mountain View, CA (US)

(21) Appl. No.: 18/007,938

(22) PCT Filed: Jun. 4, 2021
(86) PCT No.: PCT/US2021/035817

§ 371 Date: Dec. 2, 2022

Related U.S. Application Data
(60) Provisional application No. 63/035,477, filed on Jun. 5, 2020.

(51) Int. Cl. C12Q 1/6883; C12Q 1/6858; C12Q 1/6869

(52) U.S. Cl. CPC C12Q 1/6883; C12Q 1/6858; C12Q 1/6869; C12Q 2600/156; C12Q 2600/16

(57) ABSTRACT

The invention relates to polynucleotide probes, with each polynucleotide probe comprising two perfectly complementary strands. In some embodiments, each one of the strands comprises, in a 5′ to 3′ direction, a) a first target hybridization sequence, b) a first digital tag sequence, c) a first Halo barcode sequence, d) a first Halo amplification primer sequence, e) a reverse second Halo amplification primer sequence, f) a reverse second Halo barcode sequence, g) a reverse second digital tag sequence, and h) a reverse second target hybridization sequence. The invention also relates to methods of using these novel probes in to determine the levels of a minor population of DNA amongst a mixture of DNA from two different sources.

### BACKGROUND OF THE INVENTION

### Field of the Invention

The present invention generally relates to novel probes and methods of assessing circulating cell-free nucleic acid to diagnose the rejection or acceptance of a transplant.

### Background of the Invention

Early diagnosis of allograft rejection is a critically important component of post-transplant patient care. The post-transplant surveillance of organ health and rejection events is critical to the long-term success of the transplant. Early detection of any rejection event can lead to effective intervention to prevent rejection or minimize injury to the recipient.

Current transplant monitoring techniques involve expensive and invasive procedures. Graft biopsy is still the golden standard for most organ transplant monitoring. For example, endomyocardial biopsy is commonly used in cardiac allograft monitoring. It is an invasive procedure that requires routinely obtaining small samples of heart muscle for detecting rejection of a donor heart following cardiac transplantation.

In light of the biopsy complications, there have been considerable efforts to develop noninvasive techniques that might replace or reduce the need for graft biopsies. An example of such efforts is monitoring the recipient’s immune response to detect the onset of rejection. However, this approach has been criticized for producing low positive predictive values. Another method of monitoring is the assessing levels of donor DNA in the recipient’s blood. But this method of monitoring has significant limitations because it involves the use of high throughput sequencing platforms to detect minute amounts of a minor population of DNA amongst a mixture of DNA from two different sources. Thus, there is a need in the art for alternative yet reliable, reproducible, and noninvasive method for monitoring and early diagnosing allograft rejection.

The present invention provides novel double-stranded (ds) polynucleotide probes with reduced secondary structures and control for sample contamination, and without the need for endonuclease digestion to assess circulating levels of donor DNA as a way to monitor possible graft rejection or to monitor transplant organ health in a non-invasive manner. In addition, the present invention provides novel methods for determining the consensus sequence of an allele in a mixture DNA sample and methods for determining fraction of donor DNA in the mixture sample, i.e., a heterogeneous sample, without the need to genotype the DNA of the donor or recipient. The polynucleotide probes and methods provided herein can also be used in assessment of minimal residual disease (MRD) or chimerism testing, also referred to as engraftment analysis, in patients who have received a hematopoietic stem cell transplant.

### SUMMARY OF THE INVENTION

The invention relates to polynucleotide probes, with each polynucleotide probe comprising two perfectly complementary strands. In some embodiments, each one of the strands comprises, in a 5′ to 3′ direction, a) a first target hybridization sequence, b) a first digital tag sequence, c) a first Halo barcode sequence, d) a first Halo amplification primer sequence, e) a reverse second Halo amplification primer sequence, f) a reverse second Halo barcode sequence, g) a reverse second digital tag sequence, and h) a reverse second target hybridization sequence.

The invention also relates to methods of using these novel polynucleotide probes to amplify a target polynucleotide sequence present in a sample, with the methods comprising: a) denaturing the perfectly complementary strands of the polynucleotide probes provided herein to produce a first and second single stranded polynucleotide probe, b) denaturing the target polynucleotide sequence present in the sample to produce a first and second single-stranded target polynucleotide sequences, c) hybridizing each of the first and second single-stranded polynucleotide probes to the first and second single-stranded target polynucleotide sequences, respectively, wherein the single-stranded probes hybridize to the single-stranded target polynucleotide sequence in such a manner as to create circular hybrid polynucleotides, wherein the target hybridization sequences on the single-stranded polynucleotide probes are separated on the single-stranded target polynucleotide sequence, when hybridized thereto, by a gap of at least 2 nucleotides in length, d) polymerizing with nucleotides in a 5′ to 3′ direction to fill in the gap of the at least 2 nucleotides to produce a single-stranded circular probe, and e) amplifying the single-stranded circular probe without cleaving the single-stranded circular probe, wherein amplification only occurs if the gap of at least 2 nucleotides is filled during the polymerization step.

In other aspects, the present invention relates to methods for determining a consensus sequence of at least one allele of a genetic variation of DNA in a sample obtained from a transplant recipient, which contains at least the recipient DNA. In some embodiments, the method comprises: a) receiving a forward DNA sequencing read and a reverse DNA sequencing read, wherein each of the DNA sequencing reads comprises: i) a first Halo barcode sequence and a second reverse Halo barcode sequence, ii) a first digital tag sequence and a second reverse digital tag sequence, iii) a target polynucleotide sequence, wherein the target polynucleotide sequence is known to be bi-allelic, and wherein the alleles are a non-single nucleotide polymorphism (SNP) genetic variation, and iv) at least one index sequence; b) assigning the forward and reverse sequencing reads sharing the same index sequence to a single transplant recipient by mapping the index sequences to a reference index sequence, thereby producing one or more read clusters for the single transplant recipient, wherein each of the one or more read clusters comprises the forward and reverse target sequencing reads; c) verifying that the forward and reverse target sequencing reads are from the same sample preparation by confirming the sequence identity of the first and second reverse Halo barcode sequences; d) concatenating the first digital tag sequence and the second reverse digital tag sequence from each of the target sequencing reads in the read cluster to produce a long digital tag; e) identifying validated forward and reverse target sequencing reads in the read cluster by comparing the sequence of the long digital tag to a reference long digital tag sequence to confirm that there are no more than 2 mismatches between long digital tag and the reference long digital tag; f) aligning each of the validated forward and reverse target sequencing reads to target reference sequences, wherein the target reference sequences comprises one major allele of the non-SNP genetic variation or one minor allele of the non-SNP-genetic variation; and g) generating a consensus sequence for the at least one allele for the target sequence for each of the one or more read clusters. In some embodiments, the methods for determining consensus sequences can be applied to determine a consensus sequence from single DNA sequencing reads.

The invention also relates to methods of determining a donor fraction of DNA in a sample obtained from a transplant recipient comprising at least recipient DNA. In some embodiments, the DNA comprises cell-free DNA. In some embodiments, the method comprises: a) identifying a subset of informative markers, selected from a pre-determined master set of informative markers, wherein each of the markers within the master set of markers is known to be bi-allelic and wherein the allele in the bi-allelic pair is a non-single nucleotide polymorphism (SNP) genetic variation, wherein the identification of the subset of informative markers comprises: i) determining the polynucleotide sequence of all of a target set of polynucleotide sequences in the sample, wherein the target sequences correspond to the master set of informative markers, ii) determining a sample minor allele frequency of each of the master set of genetic markers within the sample, and iii) identifying the subset of informative markers based on the sample minor allele frequency in the sample being equal to or greater than 0.05%; b) estimating an initial probability of observing the genotype of each of the informative markers in the sample, based on an accepted frequency of each allele of the informative markers across a population of individuals, c) calculating an initial donor faction estimate of DNA from the estimated initial probabilities of observing the frequency of the sample minor alleles, d) calculating a conditional probability of observing the frequency of the sample minor allele from the calculated initial donor faction estimate and the standard deviation of an observed frequency of the sample minor alleles, e) applying a mixture model algorithm to the calculated initial donor faction estimate to provide an updated donor faction estimate of DNA in the sample, wherein steps (c)-(d) are repeated using the updated donor fraction of DNA in place of the initial donor faction estimate of DNA until the absolute value of the change in the updated donor faction estimate is less than a pre-set threshold value.

### BRIEF DESCRIPTION OF THE DRAWINGS

FIG. 1A depicts an exemplary procedure for forming of the double-stranded polynucleotide probes. FIG. 1B is a schematic illustration of the double-stranded probe. THS: Target Hybridization Sequence; DTS: Digital Tag sequence; HBS: Halo Barcode sequence; HAS: Halo Amplification Primer Sequence. FIG. 1C shows an exemplary sequence of the double-stranded probe with the restriction sites on both ends. The asterisks indicate the restriction enzymes cleaving sites. FIG. 1D shows the denatured probe hybridized to the forward top target polynucleotide sequence via its Target Hybridization Sequence on the right (THS1) and Target Hybridization Sequence on the left (THS2). FIG. 1E shows the denatured probe hybridized to the reverse bottom target polynucleotide sequence via its THS1 and THS2 sequences.

FIG. 2 shows a general workflow of the Spacer Multiplex Amplification ReacTion (SMART) assay using a commercially available sequencing platform.

FIG. 3 illustrates the annealing and extension steps of the SMART assay.

FIG. 4A illustrates the linearization of the circular molecule with a set of at least four forward staggered amplification primers and four reverse staggered amplification primers that hybridize to the Halo Amplification Primer Sequences in the circular molecule. FIG. 4B illustrates the resulting linear molecules.

FIG. 5A shows an exemplary sequencing reaction. FIG. 5B is a schematic illustration of the sequencing reaction product, which is the sequencing template for downstream sequencing runs.

FIG. 6 shows an exemplary sequencing readout. The sequencing reads are then proceed to be analyzed.

FIG. 7 illustrates the bioinformatics data analysis workflow including 3 stages: primary analysis, secondary analysis and tertiary analysis.

FIGS. 8 shows the allele background level estimated from 48 samples including replicates from 7 DNA specimen. LOB refers to Limit of Blank. FIG. 8B shows the LoB for 59 samples including repeats from 12 pure DNA. All samples were processed with a 192 probe set. For each sample, only homozygous targets were used to calculate the background level. Targets with less than 1000X read coverage were excluded. Top 5% homozygous targets were removed from calculation. No baseline level was subtracted from background levels for each target. The mean of the background levels for all qualifying targets were calculated for each sample. The LOB was 0.0042%, which was calculated as LoB = Mean_{blank} + 1.645(SD_{blank}).

FIG. 9A shows an exemplary result of the correlation between the expected donor fraction in the mixture sample and the estimated donor fraction. FIG. 9B shows that there is a negative correlation between the Coefficient of Variation (CV) and the target donor fraction.

FIG. 10 shows another example of using SMART method to generate probes for 192 targets for bi-allelic genetic variation. Mixtures of DNA from two DNA samples were prepared to simulate chimerism. The tested mixture levels ranged from 8% to 0.125%. Combinations of 10 pairs of target DNA were tested by randomized mixing of samples. Mixture levels were estimated in each of three replicates. If the genotypes of the donors are known, the analysis is easier for samples containing 2 or more genomes. However, the methods used herein do not require the knowledge of the donor’s genotype. FIG. 10 shows the sensitivity of detection of the donor fraction in the mixed samples. As FIG. 10 shows, there is a very good agreement between expected and observed donor fractions. As the donor fraction decreases, variation of estimation increases. As sequencing depth increases, more markers can be used to increase confidence of donor estimate.

FIGS. 11A and 11B show an example of detecting cross-contamination using the Halo Barcode Sequences as “sample identifiers.” The Halo Barcode Sequences were built into the probes for contamination detection and protection. Sample Identification features, i.e., the Halo Barcode Sequences, in the probes included prior to capturing the amplified target polynucleotide sequences for sequencing can distinguish contamination after capture since it will have a different barcode if it is a contaminant. During the analysis, all signals with the incorrect Halo Barcode sequences can be removed as contaminants, or the entire sample can be discarded. As shown in FIG. 11A, in some samples, the donor estimate has wide variation between triplicates. An example is indicated in the circle. FIG. 11B shows that, after the elimination of cross contamination using the sample identifiers, the CV significantly decreased.

### DETAILED DESCRIPTION OF THE INVENTION

### Polynucleotide Probes

The invention relates to polynucleotide probes, with each polynucleotide probe comprising two perfectly complementary strands. In some embodiments, each one of the strands comprises, in a 5′ to 3′ direction, a) a first target hybridization sequence, b) a first digital tag sequence, c) a first Halo barcode sequence, d) a first Halo amplification primer sequence, e) a reverse second Halo amplification primer sequence, f) a reverse second Halo barcode sequence, g) a reverse second digital tag sequence, and h) a reverse second target hybridization sequence.

As used herein, the term “polynucleotide” is used as it is the art and refers to a polymer of nucleotides. The polynucleotides of the present invention can be any shape, including but not limited to, linear, partially linear, circular, partially circular, nicked, branched, or helical spiral. The polynucleotides of the present invention encompass polymers comprising any numbers of nucleotides. The polynucleotides of the present invention can comprise one or more strands of the polymers of nucleotides. In one embodiment, the polynucleotides of the present invention are single-stranded (ss). In one embodiment, the polynucleotides of the present invention are double-stranded (ds). In a specific embodiment, the polynucleotides used in the present invention are DNA. In a specific embodiment, the polynucleotides used in the present invention are RNA.

The term “probe” refers to a polynucleotide that contains one or more target hybridization sequences that, when the probe is or becomes single-stranded, specifically hybridize to a target polynucleotide sequence. In some embodiments, the polynucleotide probe is single-stranded and is at least about 10 nucleotides long, and can be between about 10 and about 2000 nucleotides, or even longer. In more specific embodiments, the polynucleotide probe is single-stranded and is about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1500, or about 2000 nucleotides long. In certain embodiments, the polynucleotide probe is single-stranded and is about 150, about 175, about 200, about 250, about 275, or about 300 nucleotides long.

In some embodiments, the polynucleotide probe is a double-stranded (ds) probe comprising two complementary strands. In some embodiments, the polynucleotide probe double-stranded and is about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1500, or about 2000 base-pairs (bp) long. In certain embodiments, the polynucleotide probe is double-stranded and is about 150, about 175, about 200, about 250, about 275, or about 300 bp long. In certain embodiments, the polynucleotide probe is double-stranded and is about 150, about 175, about 200, about 250, about 275, or about 300 bp long. In an exemplary embodiment, the polynucleotide probe is double-stranded and is about 200 to about 240 bp long. In some embodiments, the probe is double-stranded with each strand being perfectly complementary to one another. However, the polynucleotide probes may be considerably longer than these examples. It is understood that any length between or within the above enumerated length or otherwise supported by the specification, including the tables, figures, and Sequence Listing, may be used. When the probe is double-stranded, e.g. a ds-DNA probe, the probe can be melted using standard temperature manipulation techniques to produce one or two single-stranded probes.

The terms “complementary” and “complementarity” are used as they are in the art and refer to the natural binding of polynucleotides by base pairing. The complementarity of two polynucleotide strands is achieved by distinct interactions between nucleobases: adenine (A), thymine (T) (uracil (U) in RNA), guanine (G), and cytosine (C). Adenine and guanine are purines, while thymine, cytosine, and uracil are pyrimidines. Both types of molecules complement each other and can only base pair with the opposing type of nucleobase by hydrogen bonding. For example, an adenine can only be efficiently paired with a thymine (A=T) or a uracil (A=U), and a guanine can only be efficiently paired with a cytosine (G=C). The base complement A=T or A=U shares two hydrogen bonds, while the base pair G≡C shares three hydrogen bonds. The two complementary strands are oriented in opposite directions, and they are said to be antiparallel. For another example, the sequence 5′-A-G-T 3’ binds to the complementary sequence 3′-T-C-A-5′. The degree of complementarity between two strands may vary from complete (or perfect) complementarity to no complementarity. The degree of complementarity between polynucleotide strands has significant effects on the efficiency and strength of the hybridization between the nucleic acid strands. In some embodiments, the polynucleotide probes provided herein comprise two perfectly complementary strands of polynucleotides.

As used herein, the term “perfectly complementary” means that two strands of a double-stranded nucleic acid are complementary to one another at 100% of the bases, with no overhangs on either end of either strand. For example, two polynucleotides are perfectly complementary to one another when both strands are the same length, e.g., 100 bp in length, and each base in one strand is complementary to a corresponding base in the “opposite” strand, such that there are no overhangs on either the 5′ or 3′ end.

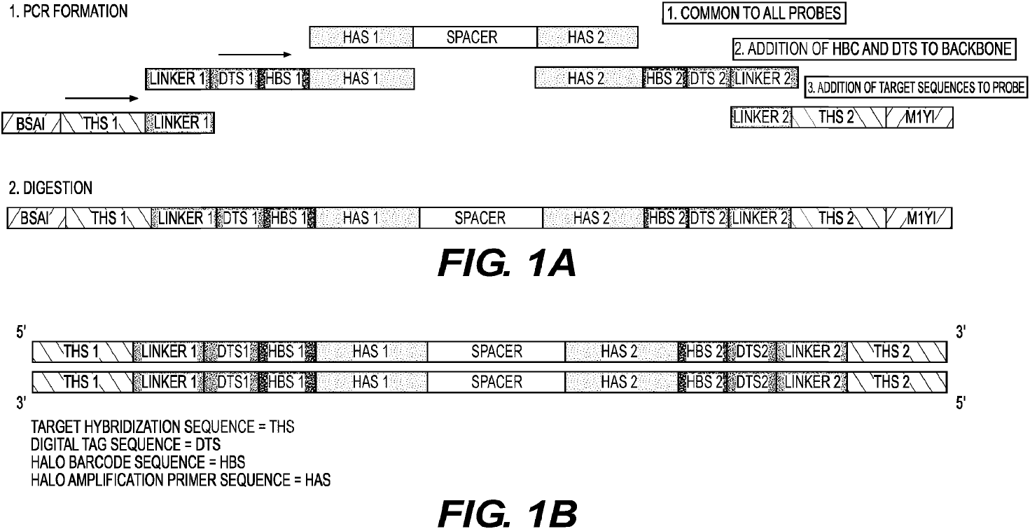

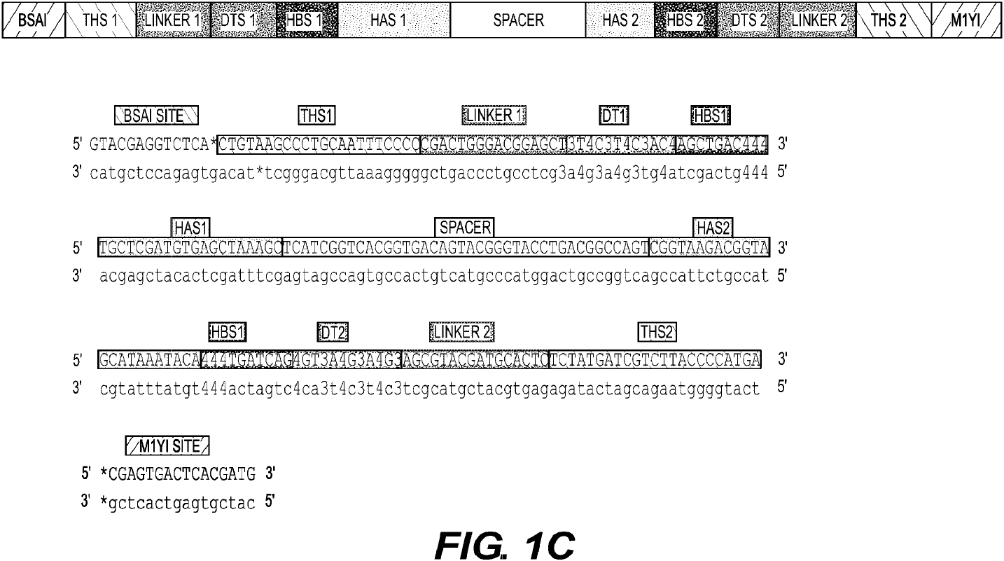

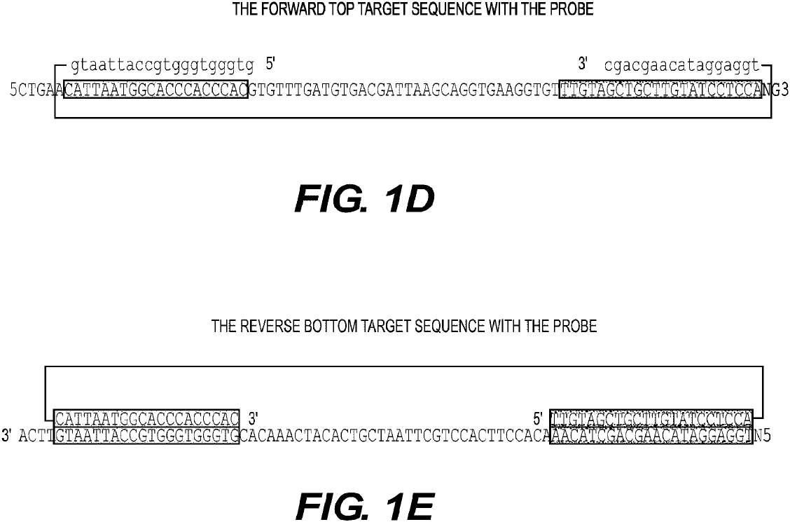

In some embodiments, each one of the two perfectly complementary strands comprises, in a 5′ to 3′ direction, a) a first target hybridization sequence, b) a first digital tag sequence, c) a first Halo barcode sequence, d) a first Halo amplification primer sequence, e) a reverse second Halo amplification primer sequence, f) a reverse second Halo barcode sequence, g) a reverse second digital tag sequence, and h) a reverse second target hybridization sequence. An exemplary illustration of the double-stranded polynucleotide probes is depicted in FIG. 1B. However, a double-stranded polynucleotide probe comprising two strands that are not perfectly complementary to one another are also encompassed by the present invention.

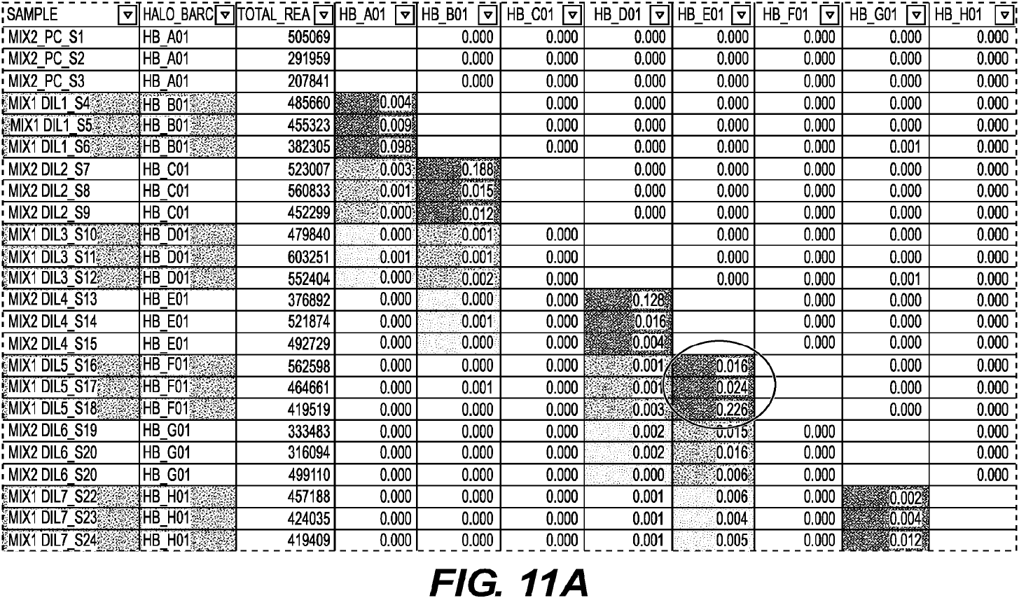

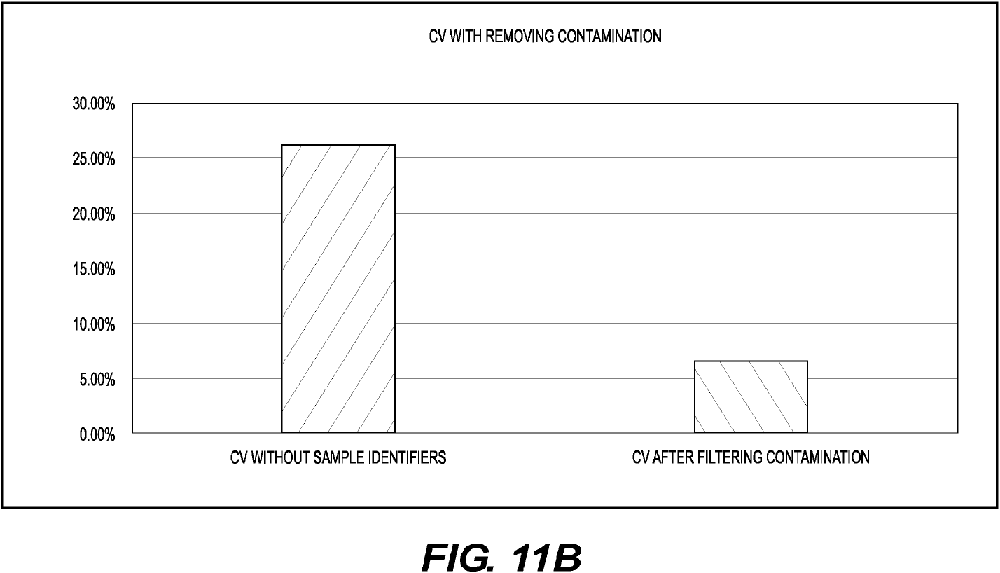

As described herein, the probes can be described as a double-stranded probe, where each single strand has the same segments. Therefore, it will be understood that, even though the probes can be double stranded, the features of the probes may be discussed in terms of the probes being single-stranded. In some embodiments, each strand of the polynucleotide probe comprises one or more Halo barcode sequences. The Halo barcode sequences are used in the probes and methods of the present invention to identify each individual sample tube for the sequencing reactions described later herein. For example, if the sequencing reactions reveal more than one Halo barcode from the same sample tube, the sample would be considered to be cross-contaminated from one or more probes from another sample tube having been introduced into the wrong sample tube. In some embodiments, the Halo barcode sequences allow the polynucleotide probes to be barcoded from the first step in the construction of the probes, as illustrated in FIG. 1A and Example 1. Thus, the information contained in the Halo barcode sequences informs the identity of the sample tube and aids in the detection cross-contamination from different sample preparations as defined herein. An example of using Halo barcode sequences to detect and eliminate cross-contamination is shown in FIGS. 11A and 11B. In some embodiments, the polynucleotide probe comprises a first Halo barcode sequence and a reverse second Halo barcode sequence. In certain embodiments, the first Halo barcode sequence and the reverse second Halo barcode sequence have identical sequences. In certain embodiments, the first Halo barcode sequence and the reverse second Halo barcode sequence are reverse complements of one another. In other embodiments, the first Halo barcode sequence and the reverse second Halo barcode sequence have different sequences from one another such that that the ds probe would contain 2 different Halo barcodes and the reverse complements thereof. In some embodiments, the Halo barcode sequences comprise artificial polynucleotide sequences. However, a polynucleotide sequence derived from a naturally occurring sequence can be used for the Halo barcode sequences. In some embodiments, the Halo barcode sequences are up to about 25 nucleotides in length. In some embodiments, the Halo barcode sequences are up to about 20 nucleotides in length. In some embodiments, the Halo barcode sequences are up to about 15 nucleotides in length. In certain embodiments, the Halo barcode sequences are up to about 12, about 11, about 10, about 9, about 8, about 7, or about 6 nucleotides in length. However, it is understood that barcode sequences outside the enumerated ranges are also encompassed by the present invention. One skilled in the art would know how to optimize the length of the Halo barcode sequences.

In further embodiments, the polynucleotide probes comprise one or more digital tag sequences. In some embodiments, the polynucleotide probe comprises a first digital tag sequence and a reverse second digital tag sequence. In some embodiments, the digital tag sequences are about 8 nucleotides to about 20 nucleotides in length. In some embodiments, the digital tag sequences are about 12 nucleotides in length. In some embodiments, the digital tag sequences comprise artificial polynucleotide sequences. In an exemplary embodiment, the digital tags next to the left primer: (SEQ ID NO: 1), and the digital tags next to the right primer:. In some embodiments, fixed nucleotides in the digital tag sequences are interlaced in the sequence as islands to prevent secondary structure (shown in boxes of SEQ ID Nos: 1 and 2). The unique design of the digital tag sequences may or may not eliminate secondary structures in the double-stranded polynucleotide probes. Moreover, the digital tag sequence are specific for each probe. In other words, the sequence of the digital tag is used to identify each probe. To that end, the number of total possible unique digital tag sequences combining both is: (3×4×3×4×3×4)^2 = 2,985,984. The identity of the variable nucleotides are defined as IUPAC nucleotide code described in Table 1 below. In some embodiments, one skilled in the art would know how to optimize the sequence and the length of the digital tag sequences.

TABLE 1

IUPAC nucleotide code

| | |
|---|---|
| IUPAC nucleotide code | Base |
| R | A or G |
| Y | C or T |
| M | A or C |
| K | G or T |
| S | G or C |
| W | A or T |
| H | A or C or T |
| B | C or G or T |
| V | A or C or G |
| D | A or G or T |
| N | A or C or G or T |

In some embodiments, the polynucleotide probe also comprises one or more linker sequences. In some embodiments, the linker sequence is located between the first hybridization sequence and the first digital tag sequence. In some embodiments, the linker sequence is located between the reverse second hybridization sequence and the reverse second digital tag sequence. In some embodiments, the linker sequence is used to incorporate the target hybridization sequence into the rest of the double-stranded polynucleotide probe. The linker sequences can be of any length. The linker sequences are generally short sequence that serve to connect functional segments of the probes. In some embodiments, the linker sequences are about 4 nucleotides to about 40 nucleotides in length. In some embodiments, the linker sequences are about 8 nucleotides to about 20 nucleotides in length. In some embodiments, the linker sequences are about 16 nucleotides in length. In some embodiments, the linker sequences comprise artificial polynucleotide sequences. In other embodiments, the linker sequences comprise polynucleotide sequences derived from a naturally occurring sequence.

In some embodiments, the polynucleotide probe further comprises a spacer sequence. The spacer sequences generally serve to lengthen the probe. The sequence of the spacer segment of the probes is not relevant to the compositions or methods of the invention. In certain embodiments, the spacer sequences are located in between the first Halo amplification primer sequence and the reverse second Halo amplification primer sequence. The space sequences can be various lengths suitable for the use. For example, in some embodiments, the spacer sequences are less than 10 nucleotides in length. In some embodiments, the spacer sequences are more than 40 nucleotides in length. In some embodiments, the spacer sequence can be more than 100 nucleotides in length. In certain embodiments, the spacer sequences are between 10-40 nucleotides in length. The optimal length of the spacer sequence can be determined by a person skilled in the art to accommodate the particular use. The space sequence can also be derived from various origin, synthetic, or a mixture of sequences derived from any origin and synthetic sequences. In one embodiment, the spacer sequences are derived from human polynucleotide sequences. In another embodiment, the spacer sequences are a non-human polynucleotide sequences. In yet another embodiment spacer sequence is a bacterial-derived polynucleotide sequence.

In some embodiments, the first target hybridization sequence and the reverse second target hybridization sequence are configured to hybridize to a single target polynucleotide sequence. In one embodiment, the first target hybridization sequence and the reverse second target hybridization sequence should be non-complementary, i.e. not hybridize to one another.

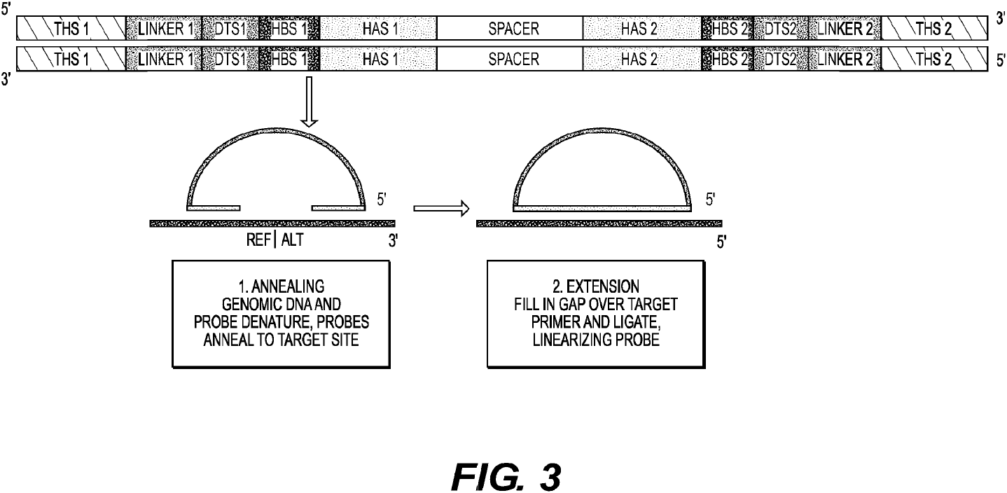

The term “target hybridization sequence” as used herein refers to polynucleotide sequences that are complementary to the sequence adjacent that is 5′ to a target polynucleotide sequence. In some embodiments, the polynucleotide probes of the present invention comprise the first target hybridization sequence and the reverse second target hybridization sequence in a 5′ to 3′ direction. A “first target hybridization sequence” is a polynucleotide sequence on the probe that is the complement sequence to a first sequence adjacent that is 5′ to the target sequence. A “reverse second target hybridization sequence” is a polynucleotide sequence on the probe that is the reverse complement sequence to a second sequence adjacent that is 3′ to the target polynucleotide sequence. Thus, the polynucleotide probes of the present invention can hybridize in two locations to a single stranded DNA that contains the target sequence, wherein the hybridization of the first target hybridization sequence and the second reverse target hybridization sequences brackets the target sequence. Ideally, the hybridization of the first target hybridization sequence and the second reverse target hybridization sequences to the DNA containing the target will circularize the probe such that the probe will fold back on itself. See FIGS. 1D, 1E, and 3.

In some embodiments, the first target hybridization sequence and the reverse second target hybridization sequence, when hybridized to the target polynucleotide sequence, are separated on the target polynucleotide sequence by a gap of at least 2 nucleotides in length. See FIGS. 1D, 1E, and 3. The gap, however, can be as long as a few thousands bps in length. For example, in some embodiments, the gap is about 2 to about 1000 nucleotides in length. In other embodiments, the gap is about 2 to about 800 nucleotides in length. In some embodiments, the gap is about 2 to about 200 nucleotides in length.

In some embodiments, the target polynucleotide sequence is known to have more than one allele. The term “allele” as used herein refers to one of the two or more alternative forms of a polynucleotide sequence that exists at a single locus on a chromosome in a population of individuals. An allele can occur in any region of the genome and may or may not result in phenotypic changes. The rate at which an allele occurs at a given locus in a given population is referred to as the allele frequency. It is generally known that, when working with genome scale and population scale sequences, the term reference allele refers to the allele that is found in a reference genome. Since the reference genome can be a random subject’s genome, the reference allele is not always the major allele. Further, the alternative allele refers to any allele, other than the reference allele, that is found at the same locus, and is not always the minor allele. In contrast, for any given locus that has two or more alleles, the allele that occurs more frequently than the alternative allele or alleles in a given population of individuals is called the “major allele” for that population. Similarly, the allele that occurs less frequently than the alternative allele or alleles in a given population is referred to as the “minor allele” for that population. A skilled person in the art would know how to determine the allele frequency of any specific allele. For example, allele frequencies in various ethnicity groups are published by the International Genome Sample Resource (IGSR) in the 1000 genome project, which can be found on the World Wide Web at internationalgenome.org/data-portal/sample.

In some embodiments, the alleles as used herein refer to the alternative sequences of a genetic variation. Genetic variation, as commonly known in the art, generally refers to the difference in polynucleotide sequences between individuals within a population. Common types of genetic variation include, but are not limited to, single nucleotide polymorphisms (SNPs), restriction fragment length polymorphisms (RFLP’s), short tandem repeats (STRs), variable number of tandem repeats (VNTR’s), hypervariable regions, minisatellites, repeats (including without limitation, dinucleotide repeats, trinucleotide repeats, tetranucleotide repeats, simple sequence repeats), insertions, deletions, duplication, copy number variation, translocation, and inversion, which are all contemplated by the present invention. In some embodiments, the genetic variations that are in the target polynucleotide sequences are known to be bi-allelic, which means that there are only two known alternative forms of the genetic variations. In some embodiments, the genetic variations targeted by the methods of the present invention are non-SNP genetic variations, meaning that the genetic variation in the target sequences do not comprise an SNP. As is well-known an SNP is a genetic variation in which the only difference in the genetic sequences is a single base swap. A deletion mutation of a single base over a reference sequence is not considered an SNP. Likewise, a single base insertion over a reference sequence is not considered an SNP. In some embodiments, the genetic variations targeted by the probes and methods of the present invention are not single base deletions. In other embodiments, the genetic variations targeted by the probes and methods of the present invention are not single base insertions. In some embodiments, the genetic variations targeted by the probes and methods of the present invention are single base deletions. In other embodiments, the genetic variations targeted by the probes and methods of the present invention are single base insertions. In other embodiments, the insertion or deletion genetic variations targeted with the probes and methods of the present invention comprise an insertion or a deletion of a single stretch of DNA sequence ranging from two to hundreds of base-pairs in length. In certain specific embodiments, the non-SNP genetic variation comprises insertions, deletions, variable number of tandem repeats (VNTRs), duplication, repeats, hypervariable regions, minisatellites, copy number variation, translocation, and inversion. In one specific embodiment, the non-SNP genetic variation is an insertion. In another specific embodiment, the non-SNP genetic variation is a deletion. In some embodiments, the minor allele of the non-SNP genetic variation is known to have an occurrence in a population of no lower than about 30%. In some embodiments, the minor allele of the non-SNP genetic variation is known to have an occurrence in a population of no lower than about 35%, about 40%, or about 50%. In one specific embodiment, the minor allele of the non-SNP genetic variation is known to have an occurrence in a population of about 40% to about 50%. In another specific embodiment, the minor allele of the non-SNP genetic variation is known to have an occurrence in a population of at least about 50%.

The target polynucleotide sequence encompassed by the present invention can be any region of the genome of any species. In some embodiments, the target polynucleotide sequence is a human genomic sequence. In some embodiments, the target polynucleotide sequence can be derived from any region of the human genome. In other embodiments, the target polynucleotide sequence can be derived from one or more genes implicated in a disease or condition.

Exemplary target polynucleotide sequences, including the chromosome number, the reference SNP (rs or RefSNP) number, the reference allele, and the alternative allele are provided in Table 2 below. The rs ID numbers listed in Table 2 are from the National Center for Biotechnology Information (NCBI)′s RefSNP catalog.

TABLE 2

Target polynucleotide sequences and respective reference and alternative alleles

| Chromosome | Reference SNP | Reference Allele | Alternative Allele |
|---|---|---|---|
| 1 | rs35757954 | C | CG |
| 1 | rs34947838 | CTT | C |
| 1 | rs10711813 | AG | A |
| 1 | rs3831887 | C | CTGT |
| 1 | rs78120377 | T | TTA |
| 1 | rs34437412 | T | TG |
| 1 | rs34769521 | G | GTA |
| 1 | rs144201174 | CTA | C |
| 1 | rs11305787 | AG | A |
| 1 | rs2308107 | CCTT | C |
| 2 | rs11447995 | C | CT |
| 2 | rs143083572 | C | CTA |
| 2 | rs35592149 | TA | T |
| 2 | rs67041051 | A | AATG |
| 2 | rs78147528 | A | AATC |
| 2 | rs35763561 | A | ACCG |
| 2 | rs11288645 | TG | T |
| 2 | rs35228295 | AT | A |
| 2 | rs60225969 | T | TG |
| 2 | rs5837741 | C | CT |
| 2 | rs5831852 | CT | C |
| 2 | rs11420113 | G | GC |
| 2 | rs34552285 | TG | T |
| 2 | rs34295643 | G | GCTC |
| 2 | rs11423826 | C | CA |
| 2 | rs34248428 | TC | T |
| 2 | rs57839738 | GA | G |
| 2 | rs10707086 | TA | T |
| 2 | rs111666747 | A | AAGAG |
| 3 | rs35065651 | AC | A |
| 3 | rs5854955 | C | CG |
| 3 | rs76235514 | GTTTA | G |
| 3 | rs56403747 | CTT | C |
| 3 | rs11328832 | TA | T |
| 3 | rs11293699 | TG | T |
| 3 | rs10575964 | CAT | C |
| 4 | rs33918560 | CA | C |
| 4 | rs3064677 | C | CCTGT |
| 4 | rs33979172 | TGTGGGA | T |
| 4 | rs34877992 | CA | C |
| 4 | rs35344864 | C | CACA |
| 4 | rs138177002 | TCTGA | T |
| 4 | rs36045877 | TCC | T |
| 4 | rs11355873 | TA | T |
| 4 | rs35011201 | TC | T |
| 4 | rs11447738 | C | CG |
| 5 | rs10538912 | AGT | A |
| 5 | rs5869879 | A | AC |
| 5 | rs11357877 | CA | C |
| 5 | rs112962511 | G | GTAGAA |
| 5 | rs2307656 | ATAAGT | A |
| 5 | rs11276961 | GAGTGT | G |
| 5 | rs11459112 | G | GA |
| 5 | rs67925455 | AG | A |
| 5 | rs34999014 | GTT | G |
| 5 | rs5870715 | T | TG |
| 5 | rs11396620 | G | GT |
| 5 | rs56229416 | T | TG |
| 5 | rs36113411 | A | AT |
| 5 | rs5868444 | TA | T |
| 5 | rs10648802 | G | GCTT |
| 5 | rs57811063 | C | CA |
| 5 | rs72335484 | AAGAG | A |
| 5 | rs150933905 | A | AG |
| 6 | rs5881455 | AGC | A |
| 6 | rs34247566 | T | TA |
| 6 | rs34814242 | TA | T |
| 6 | rs34806875 | C | CCT |
| 6 | rs11307065 | GT | G |
| 6 | rs112588184 | G | GTCCCACCAGT |
| 6 | rs111407306 | A | ATTTAC |
| 6 | rs112235399 | C | CT |
| 6 | rs369398614 | A | AG |
| 6 | rs11371791 | A | AT |
| 6 | rs5877334 | T | TG |
| 6 | rs34221858 | T | TCA |
| 6 | rs10683374 | T | TTAAG |
| 6 | rs9340850 | GT | G |
| 7 | rs34135791 | GC | G |
| 7 | rs5887691 | A | AG |
| 7 | rs35328933 | TA | T |
| 7 | rs141772951 | T | TTACTC |
| 7 | rs5886367 | A | AC |
| 7 | rs372833633 | C | CTT |
| 7 | rs5883032 | C | CA |
| 7 | rs5883297 | TC | T |
| 7 | rs35727040 | TA | T |
| 7 | rs3217072 | GT | G |
| 7 | rs35164988 | C | CT |
| 7 | rs10538815 | ACT | A |
| 7 | rs34482234 | CAA | C |
| 7 | rs11310530 | CT | C |
| 7 | rs79518236 | ACT | A |
| 7 | rs11387543 | T | TC |
| 8 | rs150051936 | T | TAG |
| 8 | rs146340341 | CTG | C |
| 8 | rs35679778 | A | AC |
| 8 | rs150797873 | T | TATCTA |
| 8 | rs5894360 | CCTT | C |
| 8 | rs149684303 | C | CA |
| 8 | rs71553885 | G | GTAAAATGT |
| 8 | rs66487920 | GTGAT | G |
| 8 | rs111862307 | CA | C |
| 9 | rs5898601 | C | CA |
| 9 | rs3831176 | GAGA | G |
| 9 | rs35657994 | TC | T |
| 9 | rs11279785 | GGGACT | G |
| 9 | rs68155284 | CAG | C |
| 10 | rs3036299 | CATGCA | C |
| 10 | rs60234473 | A | AG |
| 10 | rs35380125 | CG | C |
| 10 | rs36030543 | A | AT |
| 10 | rs113641236 | T | TC |
| 10 | rs35078465 | TG | T |
| 10 | rs57208782 | T | TCGGAA |
| 10 | rs3837320 | CT | C |
| 10 | rs10543311 | TAGC | T |
| 10 | rs36035443 | AT | A |
| 10 | rs55714089 | CAG | C |
| 10 | rs3087177 | G | GATTT |
| 10 | rs10665757 | T | TAGTC |
| 10 | rs149193032 | TTTG | T |
| 11 | rs148872433 | G | GAC |
| 11 | rs5793069 | TG | T |
| 11 | rs11368532 | A | AC |
| 11 | rs11380459 | C | CA |
| 11 | rs34274017 | AT | A |
| 11 | rs5795497 | TG | T |
| 11 | rs36059095 | C | CT |
| 11 | rs66682453 | TATG | T |
| 11 | rs35847449 | T | TC |
| 12 | rs5801835 | GC | G |
| 12 | rs66960151 | CTG | C |
| 12 | rs58083965 | T | TA |
| 12 | rs5801316 | T | TAGG |
| 12 | rs10708785 | AG | A |
| 12 | rs5796949 | TC | T |
| 13 | rs34624842 | ATATG | A |
| 13 | rs112335292 | G | GTCTC |
| 13 | rs3837574 | A | AAC |
| 13 | rs10559731 | AGTT | A |
| 13 | rs5806384 | TA | T |
| 13 | rs11313499 | AC | A |
| 13 | rs34077742 | C | CTTG |
| 13 | rs61044491 | C | CT |
| 14 | rs11305802 | CG | C |
| 14 | rs111541811 | T | TGCAG |
| 14 | rs34373257 | CA | C |
| 14 | rs5809571 | T | TCATGCC |
| 14 | rs142131288 | CAG | C |
| 14 | rs60425088 | A | AG |
| 14 | rs75631385 | G | GAC |
| 15 | rs10611079 | ACAT | A |
| 15 | rs5812677 | TG | T |
| 15 | rs572920671 | A | AG |
| 15 | rs55844159 | C | CT |
| 15 | rs34118372 | AG | A |
| 15 | rs35727345 | C | CT |
| 15 | rs35161309 | G | GTTC |
| 15 | rs10617653 | CTAT | C |
| 16 | rs35500682 | ACT | A |
| 16 | rs10532726 | GAGA | G |
| 16 | rs71138055 | TG | T |
| 17 | rs60858648 | T | TG |
| 17 | rs142499819 | CTGA | C |
| 17 | rs67772083 | ACTT | A |
| 17 | rs17338621 | TAA | T |
| 17 | rs11317870 | GT | G |
| 17 | rs56693861 | G | GT |
| 18 | rs10664892 | A | ACT |
| 18 | rs58337947 | G | GA |
| 18 | rs5823024 | A | AGTTAATGATT |
| 18 | rs74176746 | T | TAAGGCA |
| 18 | rs140837178 | A | AAC |
| 18 | rs35002074 | AT | A |
| 18 | rs11323350 | AT | A |
| 18 | rs35419086 | TC | T |
| 18 | rs572139561 | T | TAATC |
| 19 | rs5827388 | TC | T |
| 19 | rs34487308 | CT | C |
| 19 | rs35118932 | AACAC | A |
| 19 | rs34043164 | GGA | G |
| 20 | rs36035882 | C | CA |
| 20 | rs11086905 | T | TA |
| 20 | rs531633521 | G | GGC |
| 20 | rs34895568 | A | AG |
| 20 | rs138794861 | CTG | C |
| 20 | rs3215684 | A | AT |
| 21 | rs35967595 | A | ATGAC |
| 21 | rs11356622 | GT | G |
| 21 | rs10570819 | CACAGG | C |
| 22 | rs34521357 | A | AT |

In some embodiments, the polynucleotide probes further comprise one or more restriction enzyme sites. In certain embodiments, the polynucleotide probes comprise two restriction enzyme sites, each located on the 5′ and 3′ terminal of the polynucleotide probe. Restriction enzymes, also called restriction endonucleases, are generally known in the art. Typically, they are enzymes that cleave nucleic acids, such as DNA, at or near specific recognition sequences within the molecules known as restriction enzyme sites. Restriction enzymes recognize a specific sequence of polynucleotides and produce a double-stranded, single-stranded, or an overhang cut in the polynucleotide. Naturally occurring restriction enzymes are generally categorized into four groups (Types I, II III, and IV) based on their composition and enzyme cofactor requirements, the nature of their target sequence, and the position of their DNA cleavage site relative to the target sequence.

Restriction enzymes can be chosen based on the desired 5′ and 3′ ends of the sequence. Most flexibility may be obtained with restriction enzymes that cleave outside the recognition site, and whose restriction site is outside the desired sequence. For example, in some embodiments, the polynucleotide probes comprise one or more restriction enzyme sites for Type II restriction enzymes. However, it is understood that any restriction enzymes can be used for the purpose of the present invention. Type II restriction enzymes, which cleave nucleic acids, typically DNA, at defined positions close to or within their recognition sequences, producing discrete restriction fragments and distinct gel banding patterns. Type II restriction enzymes are a collection of unrelated proteins of many different sorts and frequently differ in amino acid sequence from one another. Most type II restriction enzymes cleave DNA within their recognition sequences and recognize DNA sequences that are symmetric because they bind to DNA as homodimers. However, some type II restriction enzymes recognize asymmetric DNA sequences because they bind as heterodimers. Some type II restriction enzymes recognize continuous sequences, while others recognize discontinuous sequences. Cleavage by type II restriction enzymes leaves a 3′-hydroxyl on one side of each cut and a 5′-phosphate on the other. Another common type II restriction enzymes, usually referred to as “type IIS restriction enzymes.” Type IIS restriction enzymes recognize asymmetric DNA sequences and cleave outside of their recognition sequence.

In certain embodiments, the polynucleotide probes of the present invention comprise restriction sites for two restriction enzymes: Bsal and Mlyl, or isoschizomers thereof. In certain embodiments, the Bsal restriction site is located on the 5′-end of the polynucleotide probe, and the Mlyl restriction site is located on the 3′-end of the polynucleotide probe, as shown in FIG. 1C. Digestion with Bsal generates a 5′ overhang five bases inward from the recognition site. Bsal recognizes the sequence GGTCTCN↓NNNN↑ (SEQ ID NO: 3) and leaves a 5′ NNNN overhang, where the arrows indicate the cleavage sites. The recognition sequence of Mlyl is GAGTC (SEQ ID NO: 4), but the restriction site is 5 bases inward (e.g., GAGTCNNNNN↑↓, SEQ ID NO: 5). Mlyl generates a blunt end with a 5′ phosphate group. N represents any nucleotide (A, T, C, or G). A skilled person in the art would readily know how to choose the appropriate restriction enzymes for the intended use.

The present invention further provides a population of polynucleotide probes disclosed herein. In some embodiments, each member of the population of polynucleotide probes comprises the same first target hybridization sequence and the same reverse second target hybridization sequence. In some embodiments, each member of the population of polynucleotide probes comprises a unique digital tag sequence and a unique reverse digital tag sequence. In one exemplary embodiment, the population of polynucleotide probes provided herein comprise up to about 10 million polynucleotide probes having the same first target hybridization sequence and the same reverse second target hybridization sequence. Each of the about 10 million polynucleotide probes has at least one unique digital tag sequence. Thus, in this specific embodiment, the population of polynucleotide probes have about 10 million different sequences due to the unique digital tag sequences although the population of about 10 million polynucleotide probes are configured to hybridize to the same single target polynucleotide sequence. In another specific embodiment, the population of polynucleotide probes provided herein comprise up to about 9 million polynucleotide probes having the same first target hybridization sequence and the same reverse second target hybridization sequence, and each of the about 9 million polynucleotide probes has a unique digital tag sequence. In some embodiments, the forward and the reverse digital tag sequences in each probe are independent from each other, i.e., the forward and the reverse digital tag sequences in each probe have different sequences. It is understood that the population of polynucleotide probes provided herein can comprise any number of polynucleotide probes depending on the application.

In some embodiments, at least two polynucleotide probes in the population have identical Halo barcode sequences and have identical reverse second Halo barcode sequences, e.g., within a given population of double-stranded probes, there would be a total of only four unique Halo barcode sequences: two on the “first strand” (a forward and reverse Halo barcode) of every probe and two reverse complements thereof (a forward and reverse Halo barcode) on the “second strand” on every probe.

The present invention also provides one or more collections of polynucleotide probes, and each collection of polynucleotide probes comprise one or more of populations of polynucleotide probes disclosed herein. In some embodiments, each population of polynucleotide probes comprised in the collection hybridizes to a different target polynucleotide sequence, e.g., if the collection comprises two populations, the first population would hybridize to a first target and the second population would hybridize to the second target. In one exemplary embodiment, the collection of polynucleotide probes comprises one population of polynucleotide probes and each member of the population comprises the same first target hybridization sequence and the same reverse second target hybridization sequence. Thus, in this embodiment, the population of polynucleotide probes hybridizes to the same target polynucleotide sequence. In other embodiments, the collection of polynucleotide probes comprises two or more populations of polynucleotide probes, and each population hybridizes to a different target polynucleotide sequence. In some embodiments, at least two polynucleotide probes in the collection have the identical Halo barcode sequence and the identical reverse second Halo barcode sequence. In other embodiments, all of the polynucleotide probes in the collection have the identical Halo barcode sequence and the identical reverse second Halo barcode sequence.

In certain embodiments, the polynucleotide probes of the present invention can be attached to a detectable label. Typical labels include, without being limited to, radioactive isotopes, radioactive phosphates, ligands, biotin, chemiluminescent agents, fluorophores, and enzymes, all of which are contemplated by the present invention.

### Method of Amplifying Target Polynucleotide Sequences

The present invention further provides methods of amplifying target polynucleotide sequences present in a sample. As used herein, the term “amplifying” or “amplification” generally refers to any method, technique, or system that can generate copies of a nucleic acid molecule. In some embodiments, the amplification occurs in the presence of four different nucleoside triphosphates and one or more polymerases or their functional variants in an appropriate buffer and at a suitable temperature. In some embodiments, the amplification involves a polymerase chain reaction (PCR) or its variation. Techniques for performing PCR are well-known in the art. Common variations of PCR include, but are not limited to, multiplex-PCR, multiplex ligation-dependent probe amplification (MLPA), variable number of tandem repeats (VNTR) PCR, asymmetric PCR, linear-after-the-exponential-PCR (LATE-PCR), long PCR, Klenow-based PCR, nested PCR, quantitative PCR, hot-start PCR, touchdown PCR, assembly PCR (also known as Polymerase Cycling Assembly or PCA), colony PCR, suicide PCR, and co-amplification at lower denaturation temperature-PCR (COLD-PCR). A skilled person would readily know how to choose and perform the suitable amplification method or system for the intended use.

As used herein, the term “polymerase” and its functional variants comprise any enzyme that can catalyze the polymerization of nucleotides or analogs into a polynucleotide strand. Typically but not necessarily such nucleotide polymerization can occur in a template-dependent fashion. Polymerases as used herein can include, without limitation, naturally occurring polymerases and any subunits and truncations thereof, synthetic polymerases, mutant polymerases, variant polymerases, recombinant, fusion polymerases, engineered polymerases, chemically modified polymerases, and any analogs, derivatives, or fragments thereof that retain the ability to catalyze such polymerization. Polymerases as used herein encompasses DNA polymerases, reverse transcriptases, and RNA polymerases. Some exemplary polymerases include, without limitation, Taq polymerase, Stoffel fragment of Taq polymerase, Amplitaq™ Gold, AccuPrime-Taq High Fidelity, KOD Hot Start, Pfu polymerase, Phusion Hot Start DNA Polymerase, and Pwo polymerase. Many polymerases are commercially available and a skilled person can choose based on the intended use.

As used herein, the term “sample” or “biological sample” generally refers to any material that is taken from its native or natural state, so as to facilitate any desirable manipulation, further processing, and/or modification. In some embodiments, the sample refers to a biological material that is taken from an organ transplant recipient. The organ transplants encompassed by the present invention include, but are not limited to, stem cell, bone marrow, heart, lung, liver, and kidney. Thus, the subject or patient from which the sample is taken and the methods performed thereon can be a stem cell, bone marrow, heart, lung, liver, or kidney transplant patient, respectively. In an exemplary embodiment, the sample comprises blood, serum, plasma, peripheral blood mononuclear cells (PBMCs), cells, tissues, biopsies, cerebrospinal fluid, bile, lymph fluid, saliva, urine, and stool. A sample can be further isolated and/or purified from its native or natural state. Alternatively, a sample can be derived from cell or tissue cultures in vitro. In some embodiments, a sample can be processed to extract a protein (e.g., antibody, enzyme, soluble protein, insoluble protein) or nucleic acids (e.g., RNA, DNA). In some specific embodiments, the sample is processed to extract cell-free nucleic acids. As used herein, nucleic acids comprise cell-free nucleic acids. In certain embodiments, the target polynucleotide sequence is comprised in a cell-free nucleic acid. Cell-free (cf) nucleic acids, also called circulating nucleic acids, are generally known in the art and have been used in multiple biomedical applications, such as cancer diagnosis. Cell-free nucleic acids as used herein comprise both cfDNA and cfRNA. In some embodiment, the sample is obtained from a transplant recipient. In certain specific embodiments, the sample comprises a DNA sample. In some embodiments, the sample contains at least the recipient DNA. In other embodiment, the sample contains a mixture of donor DNA and recipient DNA, and the donor and the recipient are unrelated. In specific embodiments, the sample obtained from a transplant recipient comprises donor-derived, cell-free DNA. In certain embodiments, the sample comprises less than about 10 ng of DNA. In other embodiments, the sample comprises about 10, about 20, about 50, about 75, about 100, about 150 ng of DNA. In other embodiments, the sample comprises more than about 150 ng of DNA. It is understood that the amounts enumerated here are examples, and any amount between the numbers listed here can be used.

In certain embodiments, the methods comprise amplifying the DNA in the sample prior to any of the following steps of the methods if the amount of the DNA in the sample is lower than a threshold. In some embodiments, the threshold of the amount of DNA in the sample is about 150 ng. In other embodiments, the threshold of the amount of DNA in the sample is about 100 ng. In still other embodiments, the threshold of the amount of DNA in the sample is about 50, about 40, about 30, about 20, or about 10 ng. It is understood that the threshold of the amount of DNA in the sample varies depending on the application of the methods, and can be determined by a person skilled in the art.

In some embodiments, the DNA in the sample can be amplified prior to any of the following steps of the methods using various methods adapted to the length of the DNA. In one exemplary embodiment, the sample comprises genomic DNA. Thus, in this embodiment, the entire genomic DNA in the sample can be amplified with various Whole Genome Amplification (WGA), including without being limited to, including Multiple Displacement Amplification (MDA), Degenerate Oligonucleotide PCR (DOP-PCR), and Primer Extension Preamplification (PEP). Polymerases suitable for amplifying the entire genomic DNA in the sample include, without limitation, Phi 29 polymerase; Bst 2.0 DNA Polymerase; Bst 2.0 WarmStart^{®} DNA Polymerase; Bst 3.0 DNA Polymerase; and Bst DNA Polymerase, Large Fragment. In another exemplary embodiment, the sample comprises cell-free DNA. Thus, in this embodiment, the entire cell-free DNA in the sample can be amplified by rolling circle amplification (RCA). It is understood that any methods for globally amplifying different DNA samples can be used in the methods.

It is understood that, as used in the methods provided herein, a sample obtained from any particular organ transplant recipient can be prepared in various forms. In an exemplary embodiment, a sample obtained from any particular organ transplant recipient can be prepared in serial dilutions. Thus, the term “sample preparation” refers to a particular preparation of a sample obtained from an organ transplant recipient. In some embodiments, a sample preparation refers to a sample derived from a specific organ transplant recipients. In other embodiments, a sample preparation refers to a particular preparation from a sample derived from a specific organ transplant recipient.

In one embodiment, the methods comprise denaturing the perfectly complementary strands of the polynucleotide probe described herein. As a result of the denaturing reaction, the complementary strands of the polynucleotide probe become two single-stranded polynucleotide probe, each called a first and a second single-stranded polynucleotide probe, respectively. In additional embodiments, the methods comprise denaturing a target polynucleotide sequence present in the sample. As a result, the target polynucleotide becomes two single-stranded target polynucleotides, each called a first and a second single-stranded target polynucleotide sequences, respectively. The two denaturing reactions can be carried out in one reaction or in separate reactions, and in any order. In one exemplary embodiment, the double-stranded polynucleotide probes are denatured in one reaction. The double-stranded target polynucleotides are denatured in another reaction. The two denaturing reactions can be done in any order or concurrently. In another exemplary embodiment, the double-stranded polynucleotide probes and the double-stranded target polynucleotides are denatured in one reaction concurrently. The optimal denaturing conditions may be the same or different for the two denaturing reactions. One skilled in the art would understand how to optimize the denaturing conditions for each or both of the denaturing reactions.

In another embodiment, the methods comprise hybridizing each of the first and second single-stranded polynucleotide probes to the first and second single-stranded target polynucleotide sequences, respectively. The hybridization depends on the sequence complementarity between the single-stranded polynucleotide probes and the single-stranded target polynucleotide sequences. The term “hybridize,” “hybridizing,” or “hybridization” as used herein refers to the process by which a polynucleotide strand anneals with a complementary strand through base pairing under defined hybridization conditions. Specific hybridization is an indication that two polynucleotide sequences share a high degree of complementarity. Specific hybridization complexes form under permissive annealing conditions and remain hybridized. Optimal hybridization conditions for annealing the polynucleotide probes to their respective complementary target polynucleotide sequences can be determined by one of ordinary skill in the art by routing experiments.

In some embodiments, the first and second single-stranded polynucleotide probes hybridize to the first and second single-stranded target polynucleotide, respectively, in such a manner as to create hybrid polynucleotides. As used herein, the term “hybrid polynucleotide” is a partially double-stranded polynucleotide where one strand of the double-stranded molecule is a single-strand from the probe and the second strand of the hybrid polynucleotide is a single-strand from the target polynucleotide. The hybrid polynucleotide will be double-stranded in two separate areas with at least one single-stranded region interrupting the two double-stranded regions. In some embodiments, the hybrid polynucleotides are circular. When the hybrid polynucleotides are circular, the two double-stranded regions are interrupted with two single-stranded regions. See FIGS. 1D, 1E and 3. The two double-stranded regions of are where the target hybridization sequences hybridize to a portion of the target polynucleotide sequence. The hybrid polynucleotide must be single stranded in a least one “gap region” between the two double-stranded regions. The single-stranded gap region, as used herein, is comprised of single-stranded target sequence and not the probe. In some embodiments, the single-stranded gap region is at least 2 nucleotides in length. In other embodiments, the single-stranded gap region can be any length from 2 nucleotides to a few thousand of nucleotides. In other embodiments, the single-stranded gap region is from of about 2 to about 1000 nucleotides in length. In certain embodiments, the single-stranded gap region is from about 10 to about 800 nucleotides in length. In certain embodiments, the single-stranded gap region is from about 2 to about 50 nucleotides in length. In some embodiments, the downstream reactions, such as ligation and/or amplification of the single-stranded circular probe, only occurs if the gap is filled during the polymerization reaction.

In additional embodiments, the methods comprise polymerizing with nucleotides, for example, in a 5′ to 3′ direction, to fill in a single-stranded gap region of the hybrid-polynucleotide to produce a continuous double-stranded region comprising the two target hybridization sequences from the probe. The single-stranded gap region from the target sequence, serves as a template for the polymerization reaction. Polymerization methods are well-known in the art.

The polymerization reaction will fill in the gap region to “connect” the opposite sides of the single-stranded probe portion of the hybrid polynucleotide, thus circularizing the single-stranded probe.

In some embodiments, a ligation reaction follows the polymerization reaction. Typical ligases may be temperature sensitive or thermostable. Exemplary temperature sensitive ligases, include, but are not limited to, bacteriophage T4 ligase and E. coli ligase. Exemplary thermostable ligases include, but are not limited to, Ampligase™, Archaeoglobus flugidus (Afu) ligase, Thermus aquaticus (Taq) ligase, Tβ ligase, Thermus thermophilus (Tth) ligase, Tth HB8 ligase, Thermus scotoductus (Tsc) ligase, TS2126 (a thermophihc phage that infects Tsc) RNA ligase, Thermus species AK16D ligase, and Pyrococcus furiosus (Pfu) ligase, or the like. The ligase as used herein includes, but not limited to, reversibly inactivated ligases and enzymatically active mutants and variants thereof.

In further embodiments, the methods comprise amplifying the single-stranded circular probes. In some embodiments, the amplification of the single-stranded circular probe does not require cleaving the single-stranded circular probe prior to the amplification reaction, unlike the other available methods in the art. In certain embodiments, the amplification of the circular single-stranded probe only occurs if the gap in the hybrid polynucleotide is filled during the polymerization and/or ligation steps.

In some embodiments, the single-stranded circular probes are amplified to prepare the molecules for sequencing. The probes and methods provided in the present invention may be adapted to any available Next Generation Sequencing (NGS) platform. Exemplary NGS platforms encompassed by the present invention include, without limitation, Illumina^{®} (Solexa) sequencing, Roche^{®} 454 sequencing, Ion Torrent sequencing, Pacific Biosciences (PacBio) RS/RS II, Macrogen, Qiagen GeneReader NGS system, SOLiD, MGI Complete Genomics sequencing platforms (including, without being limited to, DNBSEQ-T7, DNBSEQ-G400, DNBSEQ-G50, and DNBSEQ-G400), and Nanopore sequencing platforms (including, without being limited to, SmidgION, MinION, GridION, and PromethION platforms).

In some embodiments, the amplification of the single-stranded circular probe comprises the use of at least four forward staggered amplification primers and four reverse staggered amplification primers. The term “primer” is used as it is in the art and refers to an oligonucleotide that acts as a point of initiation for DNA amplification. In some embodiments, the primer used herein is a single-stranded oligodeoxyribonucleotide. A primer can be any length suitable for the use. For example, in some embodiments, the primer is about 15 to about 35 nucleotides in length. In other embodiments, the primer is about 35 to about 55 nucleotides in length. In some embodiments, the primer is about 39 to about 47 nucleotides in length. A primer can contain additional features. In some embodiments, the additional features allow for the detection, immobilization, or manipulation of the amplified product, but which do not alter the ability of the primer to serve as a starting reagent for DNA amplification. In other embodiments, the primer contains more than one region that have different sequences and/or functions. In an exemplary embodiment, a primer encompassed by the present invention comprises a primer amplification polynucleotide sequence and a primer sequencing polynucleotide sequence. In another exemplary embodiment, the primer amplification polynucleotide sequence and the primer polynucleotide sequencing sequence are separated from one another by a spacer nucleotide sequence of any length. In some embodiments, the spacer nucleotide sequence is 0, 1, 2, 3, 4, 5, 6, 7, 8, 9, or 10 nucleotides in length.

One common challenge in the present invention’s technological field involves the use of high throughput sequencing platforms. For example, the Illumina^{®} sequencing methodology is based on the reversible terminated chemistry concept, known as “sequencing by synthesis.” The accuracy of the high throughput sequencing platforms, such as the Illumina^{®} sequencing platform, is based on the ability of the software to accurately position the incorporation of each new nucleotide into the correct DNA molecule intended to be sequenced. This is challenging because the primers present on the flow cells are extremely close to one another. The ability of the software to properly recognize the correct position of each one of the millions of the molecules present on the flow cell is based on the random distribution of the molecules on the surface of the flowcell. Since each one of the 4 fluorescent nucleotides has a different color, every time the laser passed over the flow cell, a unique color pattern is generated. The software has the ability to recognize the unique patterns which are generated based on the random distribution of the molecules, and has the ability properly merge the patterns one after another and create the correct sequence of each one of the molecules present on the flowcell.

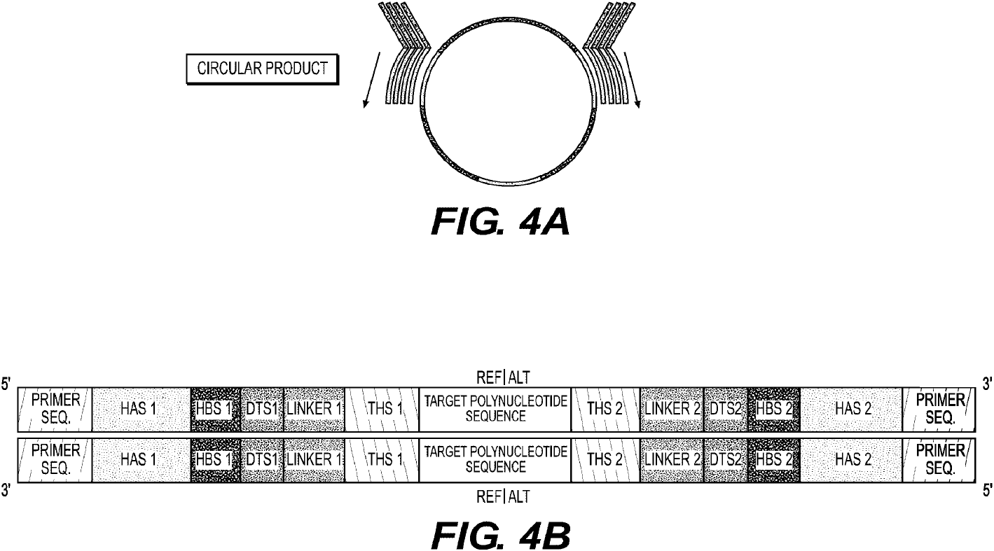

The challenge of the method provided herein is that the polynucleotide probes were built in a way that generates islands of DNA sequences, corresponding to HAS 1 and HAS 2 in FIG. 4B, which have the exact DNA sequence in all molecules at the same position. This common DNA sequence motif creates a significant challenge for the NGS, such as Illumina^{®}, sequencing software to properly de-convolute the patterns generated by the laser. Since all the molecules at the same time incorporate the same nucleotide, the sequencing software cannot properly decode the incorporation of the right nucleotide in the right molecule. The sequencing reaction will continue incorporating the same pattern every time until the sequence patterns change to random sequence patterns. However, by this time, the software has lost the memory to properly align the newly incorporated nucleotides to the right molecules. To solve this issue, a novel method of sequencing, termed dephasing strategy, is provided herein. The dephasing strategy uses a set of staggered amplification primers and keeps the randomness in the nucleotide incorporation in each cycle.

As used herein, the term “staggered primer” means a series of primers having two separate segments with a spacer nucleotide sequence of variable lengths in between, wherein the two separate segments are configured to hybridize to different targets. In some embodiment, the staggered amplification primers used in the methods described herein comprise a primer amplification polynucleotide sequence segment and a separate primer sequencing polynucleotide sequence segment. As used herein, a primer amplification polynucleotide sequence segment refers to the sequence in the staggered primer that is configured to hybridize to the Halo amplification primer sequence and a primer sequencing polynucleotide sequence segment refers to the sequence in the staggered primer that is configured to hybridize to the sequencing primer binding sequences comprised in the sequencing primers as provided herein. In the staggered primers, the primer amplification polynucleotide sequence segment and the primer polynucleotide sequencing sequence segment are separated from one another by a spacer nucleotide sequence of 0, 1, 2 or 3 nucleotides in length, such that the staggered primers have identical sequences except for the insertion of 0, 1, 2 or 3 nucleotides between the primer amplification polynucleotide sequence segment and the primer polynucleotide sequencing sequence segment.

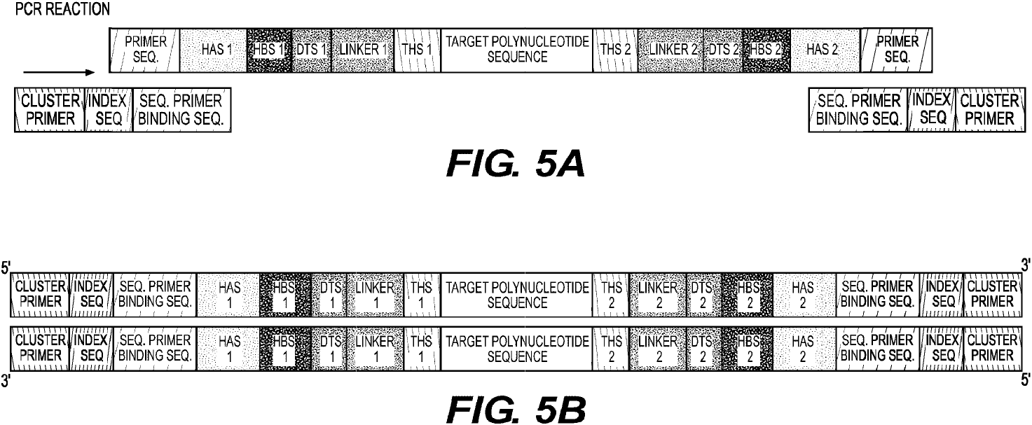

In some embodiments, the at least four forward staggered amplification primers comprise the identical primer amplification polynucleotide sequences and the identical primer sequencing polynucleotide sequences. In one exemplary embodiment, the primer amplification polynucleotide sequences are configured to hybridize to the Halo amplification primer sequence, as shown in FIG. 4A, and the primer sequencing polynucleotide sequences are configured to hybridize to the sequencing primer binding sequences comprised in the sequencing primers as illustrated in FIG. 5A. In some embodiments, the primer amplification polynucleotide sequence of the at least four forward staggered amplification primers are configured to hybridize to the first Halo amplification primer sequence of the single-stranded circular probe. In other embodiments, the primer amplification polynucleotide sequence of the at least four reverse staggered amplification primers are configured to hybridize to the reverse second Halo amplification primer sequence of the single-stranded circular probe. An exemplary illustration of the amplification of the single-stranded circular probe with staggered amplification primers is provided in FIG. 4A.

Exemplary staggered amplification primers are provided in Table 3 below. The primer amplification polynucleotide sequences are in italic, the primer sequencing polynucleotide sequences are double underlined, and the 1, 2 or 3 nucleotides between the primer amplification polynucleotide sequence and the primer sequencing polynucleotide sequences are indicated in boxes.

TABLE 3

Exemplary staggered amplification primers

| | |
|---|---|
| Primer | Sequence (SEQ ID NO) |
| Forward staggering Primer 1 | CACGACGCTCTTCCGATCTGCTTTAGCTCACATCGAGCA (SEQ ID NO: 6) |
| Forward staggering Primer 2 | CACGACGCTCTTCCGATCTAGCTTTAGCTCACATCGAGCA (SEQ ID NO: 7) |
| Forward staggering Primer 3 | CACGACGCTCTTCCGATCTCTGCTTTAGCTCACATCGAGCA (SEQ ID NO: 8) |
| Forward staggering Primer 4 | CACGACGCTCTTCCGATCTTAAGCTTTAGCTCACATCGAGCA (SEQ ID NO: 9) |
| Reverse staggering Primer 1 | GACGTGTGCTCTTCCGATCTTATCGGTAAGACGGTAGCATAAATACA (SEQ ID NO: 10) |
| Reverse staggering Primer 2 | GACGTGTGCTCTTCCGATCTGTCGGTAAGACGGTAGCATAAATACA (SEQ ID NO: 11) |
| Reverse staggering Primer 3 | GACGTGTGCTCTTCCGATCTACGGTAAGACGGTAGCATAAATACA (SEQ ID NO: 12) |
| Reverse staggering Primer 4 | GACGTGTGCTCTTCCGATCTCGGTAAGACGGTAGCATAAATACA (SEQ ID NO: 13) |

The amplification product, using the single-stranded circular probes as a template, is a linear product with primer polynucleotide sequencing sequence segments on both ends. An illustration of the linear amplification products is provided in FIG. 4B.

As a unique feature of the present invention, an exonuclease digestion is not performed at any time after the linear amplification of the single-stranded circular probe. In contrast to other methods in the art, such as the one disclosed in US Pat. No. 8,795,968, an exonuclease step is required to digest unreacted linear probes and the target DNA, and to free the circular amplified molecules from their target genomic DNA.

In additional embodiments, the methods of amplifying target polynucleotide sequences further comprise a sequencing primer amplification reaction that amplifies the linear probes using sequencing primers. An illustration of this step is provided in FIG. 5A. In some embodiments, the sequencing primers comprise, in a 5′ to 3′ direction, a cluster primer, an index sequence, and a sequencing primer binding sequence. In some embodiments, the sequencing primer binding sequences are configured to hybridize to the sequencing primers used by the NGS platform. In an exemplary embodiment, the sequencing primer binding sequences are configured to hybridize to the forward or reverse sequencing primer sequence. In some embodiments, the index sequences comprise about 5 to about 10 nucleotides. In a specific embodiment, the index sequences comprise about 7 nucleotides. The length of the index sequences can be adjusted based on the application by a skilled person in the art. In certain embodiments, the information contained in the index sequences is used to identify a sample. In an exemplary embodiment, the information contained in the index sequences is used to assign the sequence read to a specific transplant recipient, as described below. In certain embodiments, the cluster primers allow the sequences to be captured by a sequencing platform. In one exemplary embodiment, Illumina^{®} sequencing platform is used and the cluster primers allow the sequences to bind to the complementary sequences on the flow cells. It is understood that sequencing primers can be adopted to any available NGS platforms.

In further embodiments, the sequencing primer amplification reaction products, referred to as the “sequencing template” herein, can be sequenced and analyzed by an NGS platform chosen by a skilled person in the art. By way of example, a sequencing reaction using the Illumina^{®} platform is briefly described herein. In some embodiments, the sequencing templates are immobilized on the flow cell surface. Solid-phase amplification creates clusters of identical copies of each single template molecule in close proximity. The immobilized clusters of the sequencing templates are sequenced by synthesis using sequencing primers, which bind to the sequencing primer binding sequences, and four fluorescently labeled nucleotides to sequence the tens of millions of clusters on the flow cell surface in parallel, producing forward DNA sequencing reads and reverse DNA sequencing reads. In some embodiments, the forward DNA sequencing reads and reverse DNA sequencing reads can be analyzed by the method for determining a consensus sequence of at least one allele of a genetic variation of DNA in a sample obtained from a transplant recipient provided herein.

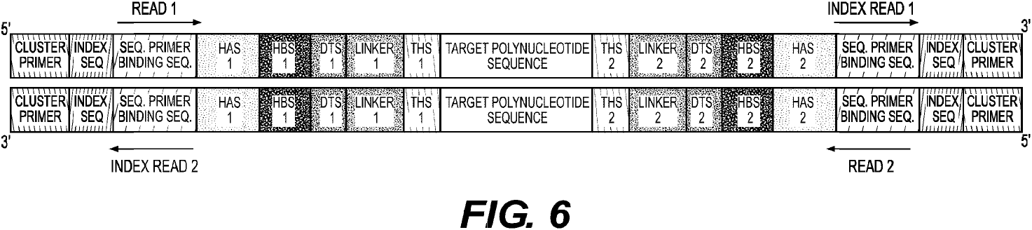

A common problem associated with the NGS platforms, such as the Illumina^{®} sequencing platform, is common static features at the same positions which lead to phasing problems during the sequencing reaction. For example, the Illumina^{®} sequencing platform defines the position of each single molecule in the flow cell during the sequencing reaction by taking advantage of the random diversity of the molecules within the flow cell. This random diversity of the molecules provides at any given cycle a very accurate position information for each molecule since the changes of near molecules to have the same incorporated nucleotide in consecutive cycles is rare. However, in the case where the Illumina^{®} sequencing primers are directly upstream of the amplification sites (e.g., the Halo Amplification Primer Sequence in FIG. 6, the sequencing reaction at the beginning of the entire flow cell produce similar sequences. As a result, the quality of the sequencing reaction will drop significantly because the sequencing instrument will have difficulties in properly identifying the accurate position of each sequencing read. The staggered amplification primers provided herein eliminate the static features at the same positions by incorporating 0, 1, 2 or 3 nucleotides between the primer amplification polynucleotide sequence and the primer sequencing polynucleotide sequences, as shown in FIG. 4A. The amplification products derived from this reaction contain a mixture of sequences starting from a random first nucleotide during the subsequent sequencing reaction steps.

Alternatively, sequencing primers with custom sequences can be used to solve the phasing problem of the NGS platforms. In one exemplary embodiment, customized sequencing primers that bind to the Halo Amplification Primer Sequences (HAS 1 and HAS 2 in the sequencing template in FIG. 6) can be used to produce the forward DNA sequencing reads and reverse DNA sequencing reads. Similarly, other customized sequencing primers can be designed by a skilled person in the art according to the specific application.

### Methods for Determining a Consensus Sequence

In other aspects, the present invention relates to methods for determining a consensus sequence of at least one allele of a genetic variation of DNA in a sample obtained from a transplant recipient, which contains at least the recipient DNA. In some embodiments, the sample is a sample obtained from a transplant recipient. In some embodiments, the sample contains at least recipient DNA. In some embodiments, the sample contains a mixture of donor DNA and recipient DNA, and the donor and the recipient are unrelated. In some embodiments, the DNA comprises cell-free DNA.

In some embodiments, the methods comprise receiving one or more DNA sequencing reads. The term “sequencing read” as used herein refers to an inferred sequence corresponding to all or part of a single DNA fragment. In some embodiments, the sequencing read is generated by a NGS platform. In some embodiments, the sequencing reads produced by the sequencing platform are converted into FASTQ files. The term “FASTQ” is used as its ordinary meaning in the art and generally refers to a text-based format for storing a biological sequence. In certain embodiments, the methods comprise receiving a forward DNA sequencing read and a reverse DNA sequencing read. However, it is understood that the methods can be adapted if only one DNA sequencing read is received. In certain embodiments, each of the DNA sequencing reads comprises: i) a first Halo barcode sequence and a second reverse Halo barcode sequence, ii) a first digital tag sequence and a second reverse digital tag sequence, iii) a target polynucleotide sequence, and iv) at least one index sequence. In some embodiments, each of the DNA sequencing reads comprises a forward index sequence and a reverse index sequence.

In some embodiments, the methods comprise assigning the forward and reverse sequencing reads sharing the same index sequence to a single transplant recipient by mapping the index sequences to a reference index sequence, thereby producing one or more read clusters for a single transplant recipient. The term “read cluster” as used herein refers to a group of related sequencing reads. For example, in some embodiments, the one or more read clusters comprise all sequencing reads of a target polynucleotide sequence. In other embodiments, the one or more read clusters are from a single transplant recipient. In certain embodiments, each of the one or more read clusters comprise the forward and reverse target sequencing reads from the same transplant recipient.

In some embodiments, the methods further comprise discarding a forward or reverse sequencing read if the index sequence comprises 3 or more mismatches compared to the reference index sequence. In some embodiments, the methods further comprise discarding a forward or reverse sequencing read if the index sequence comprises 2 or more mismatches compared to the reference index sequence. In other embodiments, the methods comprise discarding a forward or reverse sequencing read if the index sequence comprises 1 or more mismatches compared to the reference index sequence. In certain embodiments, the reference index sequences are selected from a pool of oligonucleotides about 7 bp long. In some embodiments, the reference index sequences are different from each other by at least about 3 bp. However, it is understood that the reference index sequences can have various lengths and are different from each other by a number of base-pairs. A skilled person could readily determine the appropriate configuration of the reference index sequences according to the application.

In other embodiments, each read cluster further comprises one or more index sequences used by a specific sequencing platform. As commonly known in the art, indexed sequencing allows DNA samples from multiple individuals to be pooled and sequenced together. Indexing libraries requires the addition of a unique identifier, or index sequence, to DNA samples during library preparation. For example, sequencing control software on Illumina^{®} sequencing platforms processes these tags in an automated sequencing strategy that identifies each uniquely tagged library for downstream analysis. For instance, in an exemplary embodiment, the Illumina^{®} next-generation sequencing platform is used and each read cluster further comprises i5 index read, i7 index read, or both i5 and i7 index reads. The Illumina^{®} i5 index and/or i7 index sequences are generally known in the field. In one exemplary embodiment, the Illumina^{®} index sequence libraries may comprise up to 12 unique 8-base i7 index sequences and up to 8 unique 8-base i5 index sequences. I7 sequences are applied across the columns of 96-well plate and i5 sequences are applied down the rows, thus creating up to 96 uniquely tagged libraries. During indexed sequencing, the index is sequenced in a separate read, called the Index Read, where a new sequencing primer is annealed. When libraries are dual-indexed, the sequencing run includes 2 additional reads, called the i5 and i7 index reads. In some embodiments, the reference index sequence identifies a single transplant recipient.

In some embodiments, each one of the read clusters contains the DNA sequencing reads for a single transplant recipient sharing the same index sequence. In some embodiments, the methods comprise forming a FASTQ files comprising the sequencing reads for the single transplant recipient. In other embodiments, the methods comprise forming a pair of FASTQ files, each comprising either the forward or the reverse sequencing reads, for the single transplant recipient.

In some embodiments, the methods comprise verifying that the forward and reverse target sequencing reads are from the same sample preparation by confirming the sequence identity of the first and second reverse Halo barcode sequences. In some embodiments, the methods further comprise discarding the forward and reverse target sequencing reads if the first and second reverse Halo barcode sequences comprise 1 or more mismatches to one another. In other embodiments, the methods further comprise discarding the forward and reverse target sequencing reads if the first and second reverse Halo barcode sequences comprise 2 or more mismatches to one another. In some embodiments, the Halo barcode sequences and the index sequences must identify the same transplant recipient in order for the sequencing read to be included in further processing.

In some embodiments, a sequencing quality metrics is reported for each sequencing read. In other embodiments, a sequencing quality metrics is reported for each sequencing read cluster. For example, in some embodiments, a quality score is assigned for each nucleotide base in a sequencing read. Sequencing quality scores measure the probability that a base is called incorrectly. By way of example, in the sequencing by synthesis technology, each base in a read is assigned a quality score by a phred-like algorithm. If the quality score is below a threshold, the sequencing run is failed and the sequencing reads are discarded. In some exemplary embodiments, Q30 is the percentage of the bases that have quality score at 30 or higher, and if about 70% of the bases that have quality score below 30, the sequencing run is failed and the sequencing reads are discarded. In some embodiments, each sample should be represented sufficiently in the sequencing reads. If the total number of reads assigned to certain sample is less than a threshold, the sample is excluded from analysis. In an exemplary embodiment, the threshold is about 200,000 reads. In certain embodiments, the average, median, and standard deviation of the quality scores across all sequencing reads at each position are computed and visualized. In other embodiments, the composition of nucleotide bases at each position across all sequencing reads is computed to show the uniformity of the nucleotide base in the reads. In some embodiments, a quality score is assigned to each sequencing read and the mean and standard deviation are computed and histogram is plotted for quality scores of all sequencing reads. In other embodiments, the GC content for each sequencing read is computed and the distribution of GC content for each sequencing read is plotted. In additional embodiments, the cluster density for each read cluster is calculated and reported. In some embodiments, the methods further comprises discarding low quality reads from the sequencing reads that fail a quality metrics.

In some embodiments, the methods comprise concatenating the first digital tag sequence and the second reverse digital tag sequence from each of the target sequencing reads in the read cluster to produce a long digital tag. In some embodiments, for each pair of forward and reverse sequencing reads, one digital tag is extracted from each of the forward and reverse sequencing reads. Then the two digital tags are concatenated to produce a long digital tag. In some embodiments, the first digital tag sequence or the second reverse digital tag sequence comprises between about 8 to about 20 bp. In a specific embodiment, the first digital tag sequence or the second reverse digital tag sequence comprises 12 bp. In certain embodiments, for example, first digital tag sequence and the second reverse digital tag sequence comprise 12 bp, and the resulting concatenated long digital tag is 24 bp in length.

In other embodiments, the methods further comprise identifying validated forward and reverse target sequencing reads in the read cluster by comparing the sequence of the long digital tag to a reference long digital tag sequence to confirm that there are no more than certain numbers of mismatches between long digital tag and the reference long digital tag. The number of mismatches depends on length of digital tag sequence. For example, in one embodiment, the digital tag is about 24 bp in length and no more than 2 mismatches are allowed. The number of allowed mismatches can be determined by a skilled person in the art based on the application. In some embodiments, the methods comprises discarding the forward and reverse target sequencing reads if there are 2 or more mismatches between long digital tag and the reference long digital tag.

In some embodiments, the methods comprise aligning each of the validated forward and reverse target sequencing reads to target reference sequences. In some embodiments, the methods further comprise discarding the validated forward target sequencing read and the validated reverse target sequencing read if they are not 100% complementary to each other. In some embodiments, the methods comprise discarding the validated forward target sequencing read and the validated reverse target sequencing read if they are not at least 99% complementary to each other. In other embodiments, the methods comprise discarding the validated forward target sequencing read and the validated reverse target sequencing read if they are not at least 95% complementary to each other.

In some embodiments, the target reference sequences comprise one major allele of the non-SNP genetic variation or one minor allele of the non-SNP-genetic variation. In some embodiments, the methods comprise generating a consensus sequence for the at least one allele for the target sequence for each of the one or more read clusters. In some embodiments, a consensus sequence for the target sequence for each read cluster is generated if the majority of the validated target sequencing reads in the sample align to the target reference sequences. For instance, in some embodiments, a consensus sequence for the target sequence for each read cluster is generated if greater than 50% of the validated target sequencing reads align to the target reference sequences.

In some embodiments, the methods further comprise storing the consensus sequence on a server. The term “server” is used as its meaning in the art and generally refers to any type of computer or device on a network that accepts and responds to requests made over a network. Exemplary servers include, but are not limited to, application server, cloud server, database server, dedicated server, file server, mail server, print server, proxy server, standalone server, Virtual Machine (VM) server, or web server.

In additional embodiments, the present invention also encompasses a computer-readable storage medium comprising instructions stored thereon. In some embodiments, the instructions stored on the computer-readable storage medium can be executed in a computerized system. In some embodiments, the instructions are stored in appropriate computer executable formats determined by a skilled person in the art. In some embodiments, the computerized system comprises at least one processor. In other embodiments, the instructions stored on the computer-readable storage medium can cause the at least one processor to carry out the methods provided herein. In other embodiments, some of the above functions are implemented primarily in hardware using a hardware state machine. In one exemplary embodiment, the implemented hardware is Application Specific Integrated Circuit (ASIC). In another exemplary embodiment, the implemented hardware is Field Programmable Gate Array (FPGA). Implementation of the hardware state machine so as to perform the functions described herein will be apparent to those skilled in the relevant arts.

As used herein, the computer-readable storage medium includes, without limitation, any available or later developed storage media that can be accessed by a computer and comprises volatile and nonvolatile medium, or removable and non-removable medium. By way of example, and not limitation, computer-readable storage medium can be implemented in connection with any method or technology for storage of information such as computer-readable instructions, program modules, structured data, or unstructured data. In some embodiments, the computer-readable storage medium comprises, but is not limited to, random access memory (RAM), read only memory (ROM), electrically erasable programmable read only memory (EEPROM), flash memory or other memory technology, compact disk read only memory (CD-ROM), digital versatile disk (DVD) or other optical disk storage, magnetic cassettes, magnetic tape, magnetic disk storage or other magnetic storage devices, or other tangible and/or non-transitory media which can be used to store the desired instructions. In some embodiments, the computer-readable storage medium comprises any available or later developed intangible and/or transitory media which can be used to store the desired instructions.

In other embodiments, computer-readable storage medium can be accessed by one or more local or remote computing devices for a variety of operations with respect to the instructions stored on the medium. In other embodiments, the computer-readable storage medium encompassed by the present invention can work in any computer that includes, without limitation, a personal computer, a server, a workstation, or other computer platform now or later developed.

In some exemplary embodiments, the computer program can execute some or all of the following functions: a) identifying a subset of informative markers, selected from a pre-determined master set of genetic variations according to the method provided herein; b) estimating an initial probability of observing the genotype of each of the informative markers in the sample, based on an accepted frequency of each allele of the informative markers across a population of individuals; c) calculating an initial donor faction estimate of DNA or cell-free DNA from the estimated initial probabilities of observing the frequency of the sample minor alleles; d) calculating a conditional probability of observing the frequency of the sample minor allele from the calculated initial donor faction estimate and the standard deviation of an observed frequency of the sample minor alleles, e) applying a mixture model algorithm to the calculated initial donor faction estimate to provide an updated donor faction estimate of DNA or cell-free DNA in the sample. In some embodiments, the computer program can repeat steps (c)-(d) above using the updated donor fraction of DNA or cell-free DNA in place of the initial donor faction estimate of DNA or cell-free DNA until the absolute value of the change in the updated donor faction estimate is less than a pre-set threshold value.

In additional embodiments, a computer system designed and configured to store the raw data and perform data analysis to produce report on the quality metrics and estimates of donor DNA in the sample is provided. In certain embodiments, the system consists of a computer or a server with data storage, a modem to connect to the internet, an instrument to load and read chips having array of microwells for sequencing reactions, a remote handheld or mobile device, and a software to run data analysis, generate reports, transfer, and display results to a remote handheld or mobile device.

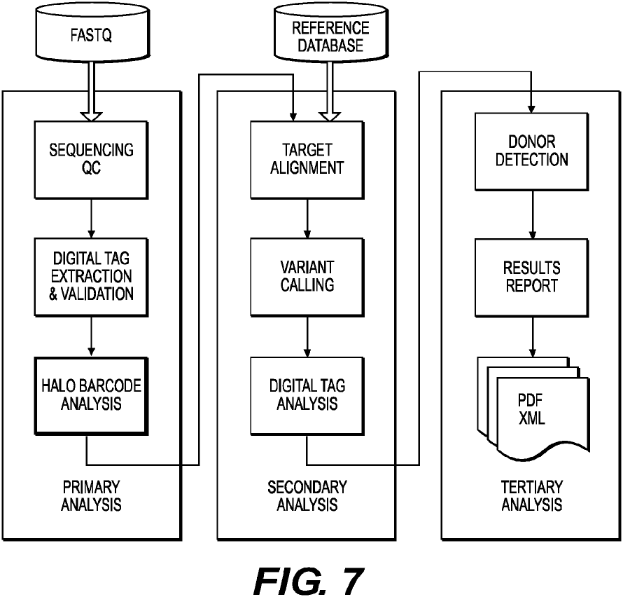

In some embodiments, the present invention encompasses a bioinformatics data analysis workflow to process the raw sequencing data and generate quality metrics for each target polynucleotide sequence and each sample (FIG. 7). In some embodiments, the bioinformatics data analysis workflow includes 3 stages: primary analysis, secondary analysis, and tertiary analysis.

In certain embodiments, the primary analysis calculates sequencing data quality metrics. In other embodiments, the primary analysis assigns reads to the sample based on the Halo barcode sequences. In yet other embodiments, the primary analysis extracts digital tag sequence for each sequencing read or each pair of sequencing reads. In some embodiments, the secondary analysis aligns the sequencing reads to reference sequences of the target polynucleotide sequences. In other embodiments, the secondary analysis calls the variants at the target locations. In still other embodiments, the secondary analysis builds consensus sequence for each read group that shared the same digital tags. In some embodiments, the tertiary analysis applies a mixture model as described herein to estimate the donor fraction. In other embodiments, the tertiary analysis generates reports on the final estimate and key quality metrics.

### Method of Determining a Donor Fraction of DNA in a Sample

The present invention further provides a method of determining a donor fraction of DNA in a sample obtained from a transplant recipient comprising at least the recipient DNA. In some embodiments, the DNA comprises cell-free DNA. In some embodiments, the method comprises identifying a subset of informative markers selected from a pre-determined master set of genetic variations. A subset of exemplary genetic variations are provided in Table 2. In some embodiments, each of the genetic variations within the master set of genetic variations are known to be bi-allelic and the allele in the bi-allelic pair is a non-single nucleotide polymorphism (SNP) genetic variation.

As used herein, the term “informative markers” refers to a pre-determined master set of genetic variations of which the sequences can be used to infer the contribution of an allele frequency from a transplant donor and a transplant recipient using a mixture model. Assuming that the genotypes of the genetic variations used in the present invention follow Mendel’s Laws of Heredity, at each bi-allelic genetic variation, four alleles can be observed. Two alleles are from the transplant recipient and two alleles are from the transplant donor. For unrelated recipient and donor, the genotypes are independent and the probability to observe each combination can be calculated based on the Mendel’s Laws of Heredity as illustrated in Table 4. In some embodiments, the transplant recipient is homozygous for each of the informative markers. In some embodiments, the transplant donor is either homozygous or heterozygous for the informative markers. In some embodiments, the transplant recipient and the transplant donor do not have the same genotype for the informative markers. In some exemplary embodiments, for unrelated recipient and donor, the informative targets can be genotypes 2, 3, 7, and 8 in Table 4 and has an expected percentage of 37.5%. In some embodiments, the recipient is homozygous for the major alleles of the informative markers. In certain embodiments, the major alleles of the informative markers have an occurrence of more than about 75% in a population. In certain embodiments, the major alleles of the informative markers have an occurrence of more than about 80% in a population.

TABLE 4

Genotypes for donor and recipient pairs with the probability of each pair

| | | | | | | | | | |
|---|---|---|---|---|---|---|---|---|---|
| Genotype (Y) | 1 | 2 | 3 | 4 | 5 | 6 | 7 | 8 | 9 |
| Recipient (R) | AA | AA | AA | Aa | Aa | Aa | aa | aa | aa |
| Donor (D) | AA | Aa | aa | AA | Aa | aa | AA | Aa | aa |
| Pr(Y) | 1/16 | ⅛ | 1/16 | ⅛ | ¼ | ⅛ | 1/16 | ⅛ | 1/16 |

In some embodiments, the identification of the subset of informative markers comprises determining the polynucleotide sequence of all of a target set of polynucleotide sequences in the sample. In some embodiments, the target set of polynucleotide sequences correspond to the master set of genetic variations. In some embodiments, the target set of polynucleotide sequences are selected from the master set of genetic variations. In some embodiments, the determining the polynucleotide sequence of all of a target set of polynucleotide sequences in the sample is performed using the polynucleotide probes provided herein. In other embodiments, the determining the polynucleotide sequence of all of a target set of polynucleotide sequences in the sample is performed using the method for determining a consensus sequence of at least one allele of a genetic variation of DNA in a sample provided herein. In some embodiments, the determining the polynucleotide sequence of all of a target set of polynucleotide sequences in the sample is performed using both the polynucleotide probes and the method for determining a consensus sequence of at least one allele of a genetic variation of DNA in a sample as provided herein.

In some embodiments, the identification of the subset of informative markers comprises determining a sample minor allele frequency (MAF) of each of the master set of genetic variations within the sample. In some embodiments, the determined sample minor allele frequency is also called observed minor allele frequency. In some embodiments, the identification of the subset of informative markers comprises identifying the subset of informative markers based on the sample minor allele frequency in the sample being equal to or greater than about 0.05%. In some embodiments, the identification of the subset of informative markers comprises identifying the subset of informative markers based on the sample minor allele frequency in the sample being less than or equal to about 20%. In other embodiments, the identification of the subset of informative markers comprises identifying the subset of informative markers based on the sample minor allele frequency in the sample being any number between about 0.05% and about 20%. However, in no case the observed minor allele frequency in the sample exceeds about 20%. In certain embodiments, if the observed minor allele frequency is less than about 0.05%, the sample is considered to only contain DNA from one source. For example, in some embodiments, if the observed minor allele frequency is less than about 0.05%, the sample is identified as not comprising donor fraction of DNA or cell-free DNA. In other exemplary embodiments, if the observed minor allele frequency is less than about 0.05%, the transplant recipient is identified as not having significant risk of transplant rejection. By way of example, in one embodiment, the master set of genetic variations comprises about 192 total genetic variations and, on average, a subset of about 35 genetic variations is identified as informative markers for sample containing DNA from unrelated recipient and donor. In some embodiments, the method comprising identifying the sample as not comprising donor fraction of DNA or cell-free DNA if the subset of informative markers comprises less than or equal to 3 informative markers.

In some embodiments, the observed MAF for informative markers are described by a mixture model. The term “mixture model” is used as its ordinary meaning in the statistics and generally refers to a probabilistic model for representing the presence of subpopulations within an overall population, without requiring that an observed data set should identify the sub-population to which an individual observation belongs. In some embodiments, a mixture model corresponds to the mixture distribution that represents the probability distribution of observations in the overall population. However, while problems associated with “mixture distributions” relate to deriving the properties of the overall population from those of the sub-populations, “mixture models” are used to make statistical inferences about the properties of the sub-populations given only observations on the pooled population, without sub-population identity information. In some embodiments, an expectation-maximization (EM) algorithm is deployed to fit observed data to the mixture model and compute donor fraction λ. An exemplary procedure of informative marker selection and model fitting is described herein. In one exemplary embodiment, a pre-determined master set of 192 bi-allelic genetic variations is used for each transplant recipient sample. For each sample, the allele frequency is calculated as the fraction of sequencing reads assigned to an allele for each of the 192 genetic variations, whether the reads are from pairs of forward and reverse sequencing reads or single sequencing reads. In alternative embodiments, the allele frequency can be calculated as the fraction of unique digital tag reads assigned to the allele. The genetic variations with minor allele frequencies in the range of [0.05%, 20%] is selected to represent the genotypes 2, 3, 7 and 8 in Table 4. As used herein, Xi represents the MAF for the ith informative marker. The set of informative markers is referred to as “I” and the number of informative markers is referred to as “N.”

In some embodiments, the method comprises estimating an initial probability of observing the genotype of each of the informative markers in the sample, based on an accepted frequency of each allele of the informative markers across a population of individuals. In certain embodiments, the accepted frequency can be determined based on the reference allele frequency information available from various publically available databases. In one exemplary embodiment, the accepted frequency can be determined based on the information in the database published by 1000 Genome Project by the International Genome Sample Resource (IGSR). A skilled person in the art would readily determine other sources for identifying allele frequencies for various populations and various genetic variations.

In some embodiments, the estimation can be performed with a Bayesian Model. The key metric to estimate is the donor fraction λ, and thus the recipient fraction is 1- λ for any bi-allelic alleles. At a particular marker Mi, the recipient’s genotype is Ri, and the donor’s genotype is Di. The allele frequency for the minor allele is Xi and Yi = (Ri; Di). The set of parameters is ⊖, which includes λ and the prior probability of the genotype at each marker.

$$Pr \left({Y}_{i}\right) = \frac{1}{16} , \text{s} y ∈ \text{3, 7}$$

$$Pr \left({Y}_{i}\right) = \frac{1}{8} , \text{s} y ∈ 2 \text{, 8}$$

The expected MAF is λ if the genotype is 3 or 7 in Table 4, where recipient and donor are both homozygous but at different alleles. The expected MAF is λ/2 if the genotype is 2 or 8 in Table 4, where the recipient is homozygous and the donor is heterozygous. The log likelihood of can be expressed as:

$$L \left(λ\right) = log Pr \left(X , Y ; θ\right) = {\sum }_{i = 1}^{N} log \left(\frac{1}{σ \sqrt{2 π}} exp \left(- \frac{{\left({X}_{i} - λ\right)}^{2}}{2 {σ}^{2}}\right)\right) + {\sum }_{i = 1}^{N} log \left(Pr \left({Y}_{i}\right)\right)$$

In some embodiments, it is assumed that the probability distribution of observed MAF Xi belongs to Exponential Family. As used herein, the term “exponential family” generally refers to a parametric set of probability distributions of a certain form. In some embodiments, the specific form can be chosen for mathematical convenience, based on, for example, useful algebraic properties. The terms “exponential class” or “Koopman-Darmois family” is sometimes used in place of “exponential family” and generally has the same meaning. The probability distribution can take the form of, without limitation, two parameter Gaussian distribution, two parameter Gamma distribution, multinomial distribution, binomial distribution, negative binomial distribution, normal distribution, exponential distribution, gamma distribution, chi-squared distribution, beta distribution, Dirichlet distribution, Bernoulli distribution, categorical distribution, Poisson distribution, Wishart distribution, inverse Wishart distribution, and geometric distribution. In some embodiments, the form of probability distribution comprises two parameter Gaussian distribution, two parameter Gamma distribution, and multinomial distribution. In other embodiments, the probability distribution of observed MAF Xi can be calculated using polynomial functions, including 1, 2, or 3 variables to a power of up to 5. Polynomial functions are generally known and are used as its ordinary meaning in the art.

In one exemplary embodiment, the initial probability of the observed MAF of all genetic variations is estimated based on Gaussian distribution. For given parameter set θ,

$$\begin{matrix}Pr \left(X , Y ; θ\right) = Pr \left(X , \left|Y ; θ\right)\right) ⋅ Pr \left(Y ; θ\right) \\ \text{s=} {\prod }_{i = 1}^{N} Pr \left({X}_{i} \left|{Y}_{i} ; θ\right)\right) ⋅ {\prod }_{i = 1}^{N} Pr \left({Y}_{i} ; θ\right)\end{matrix}$$

$$\begin{matrix}L \left(λ\right) = log Pr \left(X , Y ; θ\right) = {\sum }_{i = 1}^{N} log \left(\frac{1}{σ \sqrt{2 π}} exp \left(\frac{{\left({X}_{i} - λ\right)}^{2}}{2 {σ}^{2}}\right)\right) + {\sum }_{i = 1}^{N} log \left(Pr \left({Y}_{i}\right)\right) \\ \text{s} \frac{θ L \left(λ\right)}{θ λ} = {\sum }_{i = 1}^{N} \frac{2 \left({X}_{i} - λ\right)}{2 {σ}^{2}} \\ \text{s=} \frac{1}{{σ}^{2}} \left({\sum }_{i = 1}^{N} {X}_{i} - π λ\right)\end{matrix}$$

The log likelihood is maximized by solving:

$$\frac{θ L \left(λ\right)}{θ λ} = 0$$

$$λ = \frac{{\sum }_{i = 1}^{N} X i}{n}$$

In some embodiments, the empirical estimate is used for σ as:

$${\partial }^{3} = {{\sum }_{{X}_{i} > 3 λ / 4} \left({X}_{i} - λ\right)}^{2} / N + {\sum }_{{X}_{i} \leq 3 λ / 4} \left(2 {X}_{i} - {λ}^{2}\right) / N$$

$$\left\{\begin{matrix}{\partial }_{1} = \partial , Y ∈ \left\{3 , 7\right\} \\ {\partial }_{2} = \frac{\partial }{2} , Y ∈ \left\{2 , 8\right\}\end{matrix}\right)$$

In some embodiments, the method further comprises calculating an initial donor faction estimate of DNA from the estimated initial probabilities of observing the frequency of the sample minor alleles. In some embodiments, the genotype of the informative marker Yi is in set φ = {2; 3; 7; 8}. In some embodiments, the null hypothesis used herein is that the reference allele frequency of the genetic marker is close to 50%. Then the initial probabilities of genotype at ith informative marker is:

$$P \left(Y = 2\right) = 1 = 3 ;$$

$$P \left(Y = 3\right) = 1 = 6 ;$$

$$P \left(Y = 7\right) = 1 = 6 ;$$

$$P \left(Y = 8\right) = 1 = 3 ;$$

In certain embodiments, all the genetic variations in the pre-determined master set are selected with a reference allele frequency between 30% and 70% across various ethnicity groups based on the information available in the database published by 1000 Genome Project by the IGSR. A skilled person in the art would readily determine other sources for identifying allele frequencies for various populations and various genetic variations.

In some embodiments, the initial estimate of donor fraction is assigned as:

$${λ}^{\left[0\right]} = {max}_{i ∈ l} \left({X}_{i}\right)$$

or the 95^{th} percentile for all Xi in informative marker set. In some embodiments, the percentile can be 70^{th}, 75^{th,} 80^{th}, 85^{th,} 90^{th}, or greater. All the MAFs {Xi} can be sorted in an ascending order. In some embodiments, the percentile serves as a threshold for eliminating outliers in the input MAF values. In some embodiments, the percentile can be anything greater than the 70^{th} percentile. In some embodiment, an Xi that exceeds 95^{th} percentile is excluded from further analysis.

In other embodiments, the method comprises calculating a conditional probability of observing the frequency of the sample minor allele from the calculated initial donor faction estimate and the standard deviation of an observed frequency of the sample minor alleles.

In some embodiments, the method further comprises applying a mixture model algorithm to the calculated initial donor faction estimate to provide an updated donor faction estimate of DNA in the sample. In some embodiments, the method further comprises repeating the steps of (1) calculating a conditional probability of observing the frequency of the sample minor allele using the updated donor fraction of DNA in place of the initial donor faction estimate of DNA, and (2) updating the donor fraction estimate of DNA, until the absolute value of the change in the updated donor faction estimate is less than a pre-set threshold value. In some embodiments, the DNA comprises cell-free DNA.

In some embodiments, the calculation of the conditional probability of observing the frequency of the sample minor allele is based on the genotype of sample which contains at least the recipient DNA. In some embodiments, the calculation of the conditional probability of observing the frequency of the sample minor allele is based on the genotype of sample which contains a mixture of the recipient and the donor DNA. In some embodiments, the conditional probability of observing the frequency of the sample minor allele in the sample is calculated from the mean of a probability distribution chosen from an exponential family of the estimated initial probabilities of observing the frequency of the sample minor alleles. In an exemplary embodiment, it is assumed that the conditional probability Pr(Xi|Yi) follows a Gaussian or Gamma distribution with a mean of λ when Y=3 or Y=7, and a mean of λ/2 when Y=2 or Y=8.

$$\begin{matrix}{μ}_{1} = {λ}^{\left[i\right]} , \text{Y} ∈ \left\{3 , 7\right\} \\ {μ}_{2} = \frac{{λ}^{\left[i\right]}}{2} , \text{Y} ∈ \left\{2 , 8\right\}\end{matrix}$$

Thus, the standard deviation of Xi is calculated as:

$${\partial }^{2} = {\sum }_{{X}_{i} > 3 λ / 4} {\left({X}_{i} - λ\right)}^{2} / N + {\sum }_{{X}_{i} \leq 3 λ / 4} {\left(2 {X}_{i} - λ\right)}^{2} / N$$

$$\left\{\begin{matrix}{\partial }_{1} = \partial , \text{Y} ∈ \left\{3 , 7\right\} \\ {\partial }_{2} = \frac{\partial }{2} , \text{Y} ∈ \left\{2 , 8\right\}\end{matrix}\right)$$

Thus, in some embodiments, Xi is selected from informative marker set that has a conditional probability greater than 3λ/4 to form a subset H1. Parameter 1 (σ1) is the population standard deviation of X in H1. In other embodiments, Xi is selected from informative marker set that has a conditional probability not greater than 3λ/4 to form a subset H2. Parameter 2 (σ2) is the population standard deviation of X in H2.

In some embodiments, the form of probability distribution is selected from the group consisting of two parameter Gaussian distribution, two parameter Gamma distribution, and multinomial distribution. In one exemplary embodiment, the form of probability distribution is Gaussian distribution. Thus, the conditional probability of observing the frequency of the sample minor allele in the sample is calculated using the formulas below:

$$Pr \left({X}_{1} \left|{Y}_{1} = 3\right)\right) = \frac{1}{S} exp \left(- \frac{{\left({X}_{i} - {μ}_{1}\right)}^{2}}{2 {σ}_{1}^{2}}\right)$$

$$Pr \left({X}_{i} \left|{Y}_{i} = 7\right)\right) = \frac{1}{S} exp \left(- \frac{{\left({X}_{i} - {μ}_{1}\right)}^{2}}{2 {σ}_{1}^{2}}\right)$$

$$Pr \left({X}_{i} \left|{Y}_{i} = 2\right)\right) = \frac{1}{S} exp \left(- \frac{{\left({X}_{i} - {μ}_{2}\right)}^{2}}{2 {σ}_{2}^{2}}\right)$$

$$Pr \left({X}_{i} \left|{Y}_{i} = δ\right)\right) = \frac{1}{S} exp \left(- \frac{{\left({X}_{i} - {μ}_{2}\right)}^{2}}{2 {σ}_{2}^{2}}\right) , \text{where}$$

$$S = 2 exp \left(- \frac{\left({X}_{i} - {λ}^{2}\right)}{2 {σ}_{1}^{2}}\right) + 2 exp \left(- \frac{{\left({X}_{i} - \frac{λ}{2}\right)}^{2}}{2 {σ}_{2}^{2}}\right)$$

It is understood that other forms of probability distribution can be used. In other embodiments, the form of probability distribution can be Gamma distribution.

In some embodiments, the method comprises updating the donor faction estimate of DNA in the sample. In some embodiment, the updating comprises calculating the estimate of donor fraction using the formula:

$${λ}^{\left⌈i\right⌉} = \frac{1}{N} {\sum }_{i ∈ l} \left[{\sum }_{y ∈ \left\{3 , 7\right\}} {X}_{i} ⋅ Pr \left({Y}_{i} = y \left|{X}_{i}\right)\right) + {\sum }_{y ∈ \left\{2 , 8\right\}} 2 {X}_{i} - Pr \left({Y}_{i} = y \left|{X}_{i}\right)\right)\right]$$

$$\begin{matrix}Pr \left({Y}_{i} = y \left|{X}_{i}\right)\right) = Pr \left({X}_{i} \left|{Y}_{i} = y\right)\right) ⋅ Pr \left({Y}_{i} = y\right) / Pr \left({X}_{i}\right) \\ \text{, and} \\ \text{sPr} \left({X}_{i}\right) = {\sum }_{y ∈ Φ} Pr \left({X}_{i} \left|{Y}_{i} = y\right)\right) ⋅ Pr \left({y}_{i} = y\right)\end{matrix}$$

In other embodiments, the method comprises calculating the absolute value of the change in estimate of donor fraction using the formula:

$$\text{Δ} = \left|{λ}^{\left[t + 1\right]} - {λ}^{\left[t\right]}\right|$$

In some embodiments, the method comprises repeating the calculation of the conditional probability of observing the frequency of the sample minor allele using the updated donor fraction of DNA in place of the initial donor faction estimate of DNA until the absolute value of the change in the updated donor faction estimate is less than a pre-set threshold value. In some embodiments, the pre-set threshold value is 1.0E-6 or lower. In some embodiments, the pre-set threshold value is in the range of [1.0E-12, 1.0E-6].

It is understood that the polynucleotide probes and methods provided herein can also be used in assessment of minimal residual disease and chimerism testing. Minimal residual disease (MRD) generally refers to the presence of low-level disease detectable only by advanced laboratory testing that remains after therapy or transplantation. In some exemplary embodiments, the MRD is of B-cell acute lymphoblastic leukemia (ALL) or myeloma. In other exemplary embodiments, the MRD is of any type of blood cancer. However, it is understood that the methods can be used in assessment of any type of MRD. Chimerism testing is generally known in the art and involves identifying the donor fraction of DNA in a sample obtained from a transplant recipient of a stem cell or bone marrow transplant.

### EXAMPLES

### Example 1 - Probe Synthesis

An illustration of the double-stranded polynucleotide probe synthesis (also referred to as Spacer Multiplex Amplification ReacTion (SMART), Long Padlock Probe (LPP)) is provided in FIGS. 1A-1E. As illustrated, each strand of the double-stranded polynucleotide probe comprises, in a 5′ to 3′ direction, a first target hybridization sequence (THS 1), a linker 1, a first digital tag sequence (DTS1), a first Halo barcode sequence (HBS1), a first Halo amplification primer sequence (HAS1), a spacer, a reverse second Halo amplification primer sequence (HAS2), a reverse second Halo barcode sequence (HBS2), a reverse second digital tag sequence (DTS2), a linker 2, and a reverse second target hybridization sequence (THS 2).

An exemplary procedure for probe synthesis is illustrated in FIG. 1A. The first step in synthesizing the probe was the creation of backbone sequence that is common to all probes. The common backbone sequences comprised the first Halo amplification primer sequence, the spacer, and the reverse second Halo amplification primer sequence. In this example, the spacer sequence was only 40 nucleotides in length and the entire common backbone sequences were 82-84 nucleotides in length. Thus, the backbone sequences were chemically synthesized. It is more convenient than other probes currently available. An exemplary forward backbone sequence is shown in SEQ ID NO: 14: 5′TGCTCGATGTGAGCTAAAGCTCATCGGTCACGGTGACAGTACGGGTACCTGACGGCCAGTCGGTAAGA CGGTAGCATAAATACA 3′, (SEQ ID NO: 14), and an exemplary reverse backbone sequence is shown in SEQ ID NO: 15: 5′TGTATTTATGCTACCGTCTTACCGACTGGCCGTCAGGTACCCGTACTGTCACCGTGACCGATGAGCTT TAGCTCACATCGAGCA 3′ (SEQ ID NO: 15). The first Halo amplification primer sequence and the reverse second Halo amplification primer sequence are double underlined and the spacer sequences are in between.

Next, the digital tag sequences and the Halo barcode sequences were incorporated into the backbone. The primers comprising, in the 5′-3′ direction, the linker, the digital tag sequence, the Halo barcode sequences, and the Halo amplification primer sequences were ordered with sequences to hybridize to the first Halo amplification primer sequence and the reverse second Halo amplification primer sequence in the backbone sequences (FIG. 1A). The backbone sequences were used as templates to incorporate the linkers, digital tag sequences, and the Halo barcode sequences.

The Halo Barcode sequences were designed to have different sequences on the left (HBS1) versus the right (HBS1) in each probe, in order to prevent the probe folding onto itself and thus limiting target capture. Exemplary Halo Barcode sequences are provided in Table 5 below:

TABLE 5

Exemplary Halo Barcode Sequences

| | | |
|---|---|---|
| Barcode | HBS1 (SEQ ID NO) | HBS2 (SEQ ID NO) |
| HB_A01 | AGCTGACNNN (SEQ ID NO: 16) | CTGATCANNN (SEQ ID NO: 17) |
| HB_B01 | AGCATCTNN (SEQ ID NO: 18) | TCTACGTNN (SEQ ID NO: 19) |
| HB_C01 | ATGAGTGNNNN (SEQ ID NO: 20) | GACGTAGN (SEQ ID NO: 21) |
| HB_D01 | AGCGCTANNN (SEQ ID NO: 22) | CGATCGANNN (SEQ ID NO: 23) |
| HB_E01 | TACGAGTNN (SEQ ID NO: 24) | GTCTAGCNN (SEQ ID NO: 25) |
| HB_F01 | GATATGCNNNN (SEQ ID NO: 26) | TAGACACN (SEQ ID NO: 27) |
| HB_G01 | CGTGCTANNN (SEQ ID NO: 28) | CACGCTANNN (SEQ ID NO: 29) |
| HB_H01 | AGCTATGNN (SEQ ID NO: 30) | CGAGACGNN (SEQ ID NO: 31) |

The Halo Barcode sequences flank the Halo amplification primer sequence and the digital tag sequences.

One exemplary forward primer used in step 2 is provided as: 5′-GAGTGCATCGTACGCTVCNTVCNTVACNCTGATCANNNTGTATTTATGCTACCGTCTTACCG (SEQ ID NO: 32), and one exemplary reverse primer used in step 2 is provided as: 5′-CGACTGGGACGGAGCTVTNCDTNCDACNAGCTGACNNNTGCTCGATGTGAGC-3′ (SEQ ID NO: 33). The linker sequence is italicized, the digital tag sequence is in the box, the Halo barcode sequence is double underlined, and the Halo amplification primer sequence is bolded. The fixed bases in the digital tag sequence are interlaced in the sequence as islands to prevent secondary structure (highlighted in bold gray). In the examples provided here, each digital tag sequence is 12 nucleotides long and the patterns of the sequence are defined as IUPAC code. In this example, the total possible unique digital tag sequence combining both are (3×4×3×4×3×4)^2 = 2,985,984.

In the next step, the double stranded probes with the target Hybridization Sequence and the backbone sequence were created. To achieve that, the double-stranded PCR product previous step was amplified with PCR primers that had a Mlyl sequence engineered into the one primer, and the Bsal sequence engineered into the other primer (FIG. 1A). The products from this phase contained the restriction sites on both termini.

The restriction sites were incorporated at the ends of double stranded template by PCR amplification with engineered primers. An exemplary forward primer (with Bsal site) is provided as 5′-GTACGAGGTCTCAATGCTTGTAGCTGCTTGTATCCTCCACGACTGGGACGGAGCT-3′ (SEQ ID NO: 34), and an exemplary reverse primer (with Mlyl site) is provided as 5′-CATCGTGAGTCACTCGGTGGGTGGGTGCCATTAATGGAGTCCATCGTACGCT-3′ (SEQ ID NO: 35). This will generate the molecule shown in FIG. 1C.

Then the double-stranded PCR product was digested with restriction enzymes. The first digestion with Bsal generated a 5′ overhang five bases inward from the recognition site on the top strand, and 1 base inward on the lower strand. This created a molecule with a recessed 3′ end and a protruding 5′ end, as shown in FIG. 1C. The asterisks indicate the enzymes’ cleaving sites. The molecule was then digested with the enzyme Mlyl that cleaves 5 bases inward from the recognition site, and generates a blunt-end molecule that has a phosphate group at the 5′ end. The 5′ adenosine on the bottom strand has a terminal phosphate group after the restriction enzyme cleavage. The PCR and enzyme digestion reactions conditions following the general protocols used in the art, and one skilled in the art can readily determine the optimized conditions based on the application. The desired double-stranded polynucleotide probe is now formed (FIG. 1B). A distinguishing feature of the present invention is that an exonuclease digestion is not performed at any time after the polymerization. In contrast, other methods in the art, such as the one disclosed in US 8,795,968, require an exonuclease to digest unreacted linear probes and the target DNA, and to free the circular amplified molecules from their target genomic DNA.

An example of the primer design for SNP ID rs34769521 in Table 2 is provided herein. The forward top target sequence (double underline) is: 5′-(N)nACTGAACATTAATGGCACCCACCCAGTGTTTGATGTGACGATTAAGCAGGTGAAGGTGTTTGTAGCTG CTTGTATCCTCCANGTCTAGATATNTAGCTAATCCATACCTNTGCATNNCTATNCCTATCANTACACACTGGGGCC CANCACAGATNN-3′ (SEQ ID NO: 36). N represents a nucleotide base that can be any of A, C, G or T.

The reverse bottom target sequence (double underline) is: 3′--(N)nTGACTTGTAATTACCGTGGGTGGGTGCACAAACTACACTGCTAATTCGTCCACTTCCACAAACATCGACG AACATAGGAGGTNCAGATCTATANATCGATTAGGTATGGANACGTANNGATANGGATAGTNATGTGTGACCCCG GGTNGTGTCTANN-5′ (SEQ ID NO: 37). The sequences in the boxes are binding sites for the target Hybridization Sequences in the probes. Thus, the Target Hybridization Sequence on the right (THS1) in the final probe on top is: 5′-CATTAATGGCACCCACCCAC-3′ (SEQ ID NO: 38) and the Target Hybridization Sequence on the left (THS2) in the final probe on top is 5′-TTGTAGCTGCTTGTATCCTCCA-3′ (SEQ ID NO: 39) (FIG. 1D). In addition, the Target Hybridization Sequence on the right (THS1) in the final probe on the bottom is: 5′- GTGGGTGGGTGCCATTAATG-3′ (SEQ ID NO: 40) and the Target Hybridization Sequence on the left (THS2) in the final probe on the bottom is 5′-TGGAGGATACAAGCAGCTACAA-3′ (SEQ ID NO: 41). (FIG. 1E). The SMART assay can be used to sequence multiple, for example, more than 5000 or 10,000, target sequences in a single tube reaction.

### Example 2 - Spacer Multiplex Amplification Reaction (SMART) Assay for Illumina Sequencing

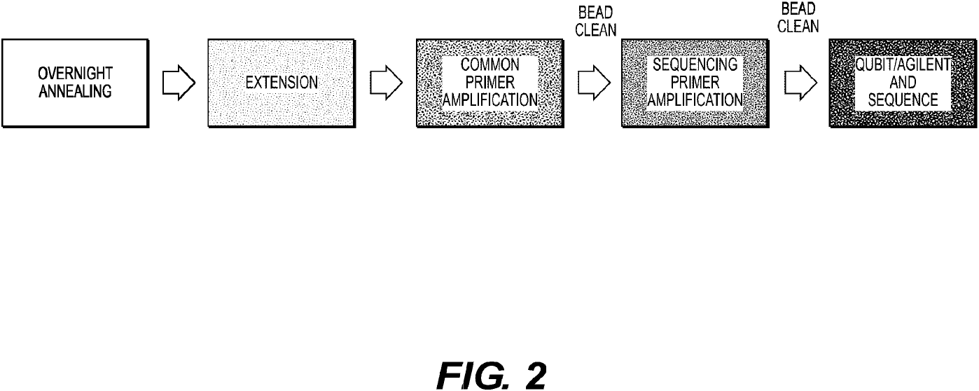

A general workflow of the SMART assay using the Illumina^{®} sequencing platform is demonstrated in FIG. 2. The first step was overnight annealing of the probes with the target sequences, followed by the extension. The next step was the amplification using common staggered primers.

The methods for PCR and ligation reactions are generally known and used in the art. In this example, 100 attomoles of probes were annealed with about 500 ng human genomic DNA. This was done by first denaturing at 95° C., then gradually decreasing temperature by 1° C. decrements to 58° C., holding for 1 minute at each temperature, and last, annealing overnight at 58° C. It is notable that, in the present invention, the probes were designed to amplify multiple target polynucleotide sequences in a multiplex fashion.

An example of the experiment is briefly described here. First, tubes or plates were place on a cold metal block on ice. After 2 minutes, about 6.5 µl of a master mix containing: 0.8 µl 10× Ampligase buffer, 5 units Ampligase™ (Epicenter^{®}), 0.5 units Stoffel fragment of Taq polymerase (Applied Biosystems), and 5.2 µl dH₂O, were added. The reaction was incubated at 58° C. for 2 minutes, and then placed on ice block. About 1.5 µl of Cold dNTP mix (1.25 mM) were added and mixed well by pipetting. Next, the reaction was incubated at 58° C. for 15 minutes and held at 37° C. An exonuclease digest was not needed after the extension.

As illustrated in FIG. 3, the single stranded probe hybridized to the target polynucleotide sequence, and was extended by a suitable DNA polymerase. It was then ligated to form a circular probe. The spacer backbone is illustrated in FIG. 3 as forming a loop between the hybridized targets. The DNA polymerase catalyzed the polymerization of DNA from the 3′ end to fill the gap between the two targets. The Ampligase™ enzyme can be used to close the circle by ligating the two ends of the probe when the enzyme reaches the 5′ end of the other target. The probes were extended and circularized using a Stoffel polymerase and Ampligase™ in the Ampligase™ buffer (Epicenter^{®}). Following circularization, no exonuclease digestion is needed.

The molecules were then linearized with a set of at least four forward staggered amplification primers and four reverse staggered amplification primers that hybridize to the Halo Amplification Primer Sequences, as shown in FIG. 4A. The entire contents of the extension and ligation reactions were used for PCR amplification in a cocktail containing 10 mM tris-HCL (PH 8.3), 50 mM Potassium Chloride, 0.25 mM Magnesium Chloride, and 2 units Amplitaq™ Gold. Exemplary forward staggered amplification primers and reverse staggered amplification primers are provided in Table 3. The cycling parameters were 10 minutes of heat inactivation at 95° C., followed by 40 cycles of 95° C. for 30 sec, 63° C. for 30 sec, and 72° C. for 30 sec. The resulting linear molecules are demonstrated in FIG. 4B. Following the linearization, no exonuclease digestion is needed. The PCR amplification products can be cleaned up using any bead-based PCR clean-up assays available in the art. Next, the PCR amplification products were used in sequencing primer amplification as shown in FIG. 5A, followed by DNA quantification and sequencing.

### Example 3 - Additional Sample Protocol

This Example further provides an exemplary protocol that was used to construct the ds-probes and amplify the captured target sequence. In brief, the workflow comprises the following steps: Creating double-stranded probes, capturing genomic DNA using probes and create probes with barcode, preparing product for sequencing sequences, and sequencing.

To create double-stranded probes, three components were assembled to create the probe according to the Tables 6-8 below.

TABLE 6

Assembly of 1 µM stock of Backbone

| | Concentration | Volume (µL) |
|---|---|---|
| Backbone #190 | 100 µM | 2 |
| Backbone #191 | 100 µM | 2 |
| Buffer | N/A | 196 |
| Total | | 200 |

TABLE 7

Assembly of 1 µM stock of Digital Tags or barcode (BC)

| | Concentration | Volume (µL) |
|---|---|---|
| LeftBC | 100 µM | 2 |
| RightBC | 100 µM | 2 |
| Buffer | N/A | 196 |
| Total | | 200 |

TABLE 8

Assembly of 5 µM stock of sequence with the Target Hybridization Sequence (THS)

| | Concentration | Volume (µL) |
|---|---|---|
| Bsa Primer | 100 µM | 5 |
| Mly Primer | 100 µM | 5 |
| Buffer | N/A | 90 |
| Total | | 100 |

In this example, a 96-well plate of double-stranded probes were prepared with a 5 µM stock dilution of the sequences with the Target Hybridization Sequences. Exemplary PCR reaction and cycling conditions are provided in Tables 9-10 below. A typical PCR cycle is about 1 hour.

TABLE 9

PCR components per probe per reaction

| Components | Concentration | Volume (µL) |
|---|---|---|
| 10X HotStar Buffer | N/A | 2.5 |
| dNTP | 10 mM | 0.5 |
| Backbone | 1 µM | 0.5 |
| Barcode or digital tag | 1 µM | 0.5 |
| Target Hybridization Sequences | 5 uM | 1.6 |
| HotStar Polymerase | N/A | 0.25 |
| dH2O | N/A | 19.15 |
| Total | | 25 |

TABLE 10

PCR Cycling Condition.

| | | |
|---|---|---|
| | Temperature | Time |
| | 95° C. | 15 min |
| 20 cycles | 95° C. | 15 sec |
| | 60° C. | 15 sec |
| | 72° C. | 30 sec |
| | 72° C. | 5 min |
| | 4° C. | hold |

PCR Products were cleaned with magnetic beads. The bead cleaning was done with 1.5X beads (37.5 µL) with 3 washes with 80% ethanol. Then the DNA was eluted in 27 µL of 10 mM Tris-HCI and 25 µL of the DNA was transferred. Following bead clean, the DNA concentration was measured by PicoGreen in triplicates. The DNA was normalized so that all probes were about the same concentration of, for example, 10-20 ng/µL.

In another version, the pooling was done prior to bead cleaning. In this example, EDTA was added right after the probe PCR. Then the DNA concentration was measured by PicoGreen in triplicates, followed by normalization and pooling before doing a bead cleaning on the combined probes. The DNA was normalized so that all probes were about the same concentration of, for example, 10-20 ng/µL. The bead cleaning was done with 1.5X beads (37.5 µL) with 3 washes with 80% ethanol. Then the DNA was eluted in 27 µL of 10 mM Tris-HCl and 25 µL of the DNA was transferred.

Enzyme digestion followed the bead cleaning as commonly performed in the art. The digestion of the probes was done with Bsal and Mlyl. Tables 11 and 12 below provide the reaction conditions for the digestion. The reaction took about 70 minutes. In some Instances, probes may have to be prepared individually.

TABLE 11

Digestion components per reaction.

| Components | Volume Probe Mix Rxn (µL) | Volume for Single Probe Rxn (µL)* |
|---|---|---|
| 10X Cut Smart Buffer | 10 | 5 |
| Probe (Mix) | 40 | 20 |
| Bsal | 2 | 1 |
| Mlyl | 2 | 1 |
| dH2O | 46 | 23 |
| Total | 100 | 50 |

^{∗}In some Instances, probes may have to be prepared individually.

TABLE 12

Enzymatic Digestion Cycling Condition.

| | |
|---|---|
| Temperature | Time |
| 37° C. | 60 min |
| 70° C. | 10 min |
| 4° C. | Hold |

Following the digestion, another bead cleaning was performed according to the method described immediately above. Next, to confirm that product size is different, about 1 µL from the probe or probe mix before digestion and after the digestion and bead cleaning were run on Agilent DNA 1000. About 30 bp difference between digested and undigested probes is expected.

Next, the probes were diluted to reach the desired target concentration for overnight annealing of the probes to genomic DNA to capture the target regions on genomic DNA. An exemplary reaction is provided in Table 13. The Cycling condition comprises incubating at 94° C. for 2 minutes, at 94° C. to 64° C. by decreasing 1° C. per cycle for 30 cycles, each cycle was 1 min. Hold at 60° C.

TABLE 13

Annealing components per reaction

| Component | Volume (µL) | Note. |
|---|---|---|
| 10X Ampligase Buffer | 0.9 | |
| Probe/Probe Mix | 2 | Calculate using spreadsheet. Have been aiming for 16 amole/µL per probe. Reaction has 32 amole per probe |
| Genomic DNA | ~ | Varies. Calculate using qPCR values. |
| dH2O | ~ | DNA + dH2O = 6.1 µL |
| Total | 9 | |

Next, the ds probes were extended using the conditions provided in Tables 14 and 15.

TABLE 14

Extension components per reaction

| Component | Volume (µL) |
|---|---|
| 10X Ampligase Buffer | 0.1 |
| Ampligase | 0.5 |
| Phusion or Q5 | 0.2 |
| 1 mM dNTP | 2 |
| dH2O | 0.2 |
| Extension mastermix/rxn | 3 |
| Annealing total rxn | 9 |
| Extension rxn total | 12 |

TABLE 15

Extension cycling condition

| | |
|---|---|
| Temperature | Time |
| 60° C. | 15 min |
| 72° C. | 5 min |
| 4° C. | Hold |

Then, the probes were amplified using primers common to all probes. Exemplary PCR reaction and cycling conditions are provide in Tables 16 and 17. The reaction cycle was about 30 minutes.

TABLE 16

PCR components per reaction

| Component | Concentration | Volume (µL) |
|---|---|---|
| Q5 Rxn Buffer | 5× | 10 |
| dNTP | 10 mM | 0.8 |
| Q5 DNA Polymerase | 2 U/µL | 0.5 |
| dH2O | - | 18.7 |
| NEB Phasers | 5 µM | 5 |
| PCR mastermix/rxn | | 35 |
| Extension and ligation Product | | ~10 |
| PCR rxn total | | 45 |

TABLE 17

Common Primer Amplification Cycling Condition

| | | |
|---|---|---|
| | Temperature | Time |
| | 98° C. | 30 sec |
| 25 cycles^{∗} | 98° C. | 10 sec |
| | 65° C. | 15 sec |
| | 72° C. | 30 sec |
| | 72° C. | 5 min |
| | 4° C. | hold |

The PCR products were cleaned with the bead cleaning. A bead cleaning was done with 1X beads by volume (45 µL) and 3 washes with 80% ethanol. Beads were eluted in 28 µL of 10 mM Tris-HCl and 25 µL was transferred. Then the DNA was eluted in 28 µL of 10 mM Tris-HCl pH8, and 25 µL of elute was taken for the following steps.

Next, the common primer amplification product was diluted at a ratio of about 1:5, and the products were amplified using common sequencing primers. Exemplary amplification reaction and cycling conditions using the NEBNext^{®} Multiplex Oligos for Illumina^{®} are provide in Tables 18 and 19.

TABLE 18

Multiplex Primer Amplification Condition

| Component | Concentration | Volume (µL) |
|---|---|---|
| Q5 Rxn Buffer | 5X | 5 |
| dNTP | 10 mM | 0.5 |
| Q5 DNA Polymerase | 2 U/uL | 0.25 |
| dH2O | - | 10.25 |
| PCR mastermix/rxn | | 16 |
| Primers | 5 uM (each) | 4 |
| CPA Clean Product | | 5 |
| PCR rxn total | | 25 |

TABLE 19

Thermal Cycler Program

| | | | | | |
|---|---|---|---|---|---|
| 1 cycle | 7 cycles | | 1 cycle | | Vol |
| Initial hold | Denaturation | Anneal/Extension | Final ext | Hold | 25 µL |
| 98° C. / 30 s | 98° C. / 10 s | 65° C. / 75 s | 65° C. / 5 min | 4° C. ∞ | |

After the sequencing amplification reaction, the products were cleaned by bead cleaning. A bead cleaning was done with 0.8X beads by volume (20 µL) and 3 washes with 80% ethanol. About 20 µL AMPure XP beads were added to the products. The DNA was eluted in 18 µL of 10 mM Tris-HCl pH 8 and 16 µL of the elute was taken. Next, the DNA was quantified by Qubit BR or Picogreen and confirmed by Agilent DNA 1000.

### Example 4 - Target Selection and Data Analysis

This Example shows an exemplary process from target selection to data analysis employed by the method provided herein.

Target Selection. A panel of genetic variations were selected from 1000 Genome Project as targets for capture. All of the genetic variations selected are non-SNP insertions or deletions across 22 autosomal chromosomes. Either a subset or the entire set of the variants form the targets were selected for capture, sequencing, and analysis. Insertion or deletions were chosen because error rates of insertions or deletions are much lower than single base substitution for both PCR and sequencing techniques, whether Sanger sequencing or Next Generation Sequencing (NGS) is used. The panel of insertion and deletion targets provides great advantage over the one of single nucleotide variants, such as SNPs. Thus, with insertion and deletion targets, noise level is significantly lower and higher signal to noise ratio can be achieved with ultra-high sensitivity.

Correction for Allele Background. The two alleles at each target can have different background. For example, if the genotype is homozygous for the reference allele for a sample, the background level for the alternative allele can be 0.1%. But when if the genotype is homozygous for the alternative allele for a sample, the background level for the reference allele can be 0.01%.

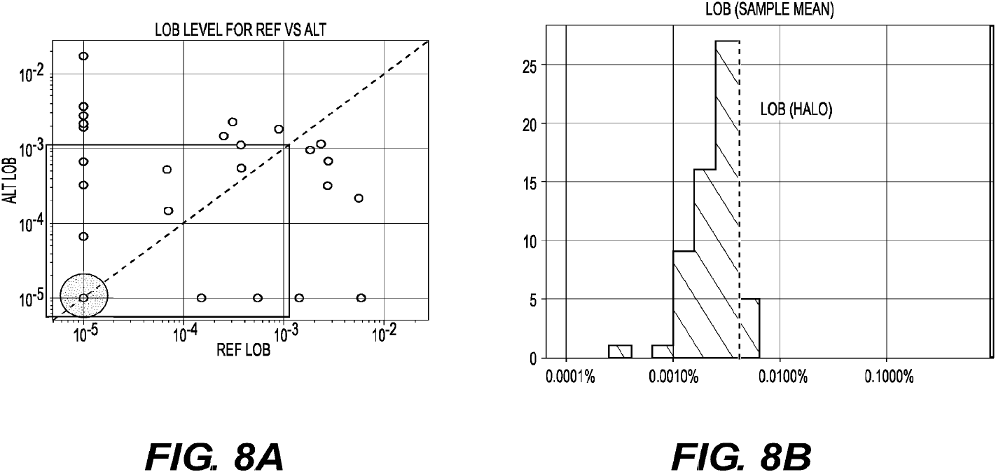

The level of background for each allele were observed consistently across samples. The background levels are pre-determined from pure DNA samples for both alleles at each target. For informative markers, the allele of the recipient as homozygous needs to be more than 75% and thus is the major allele, and the other allele is the minor allele. During the analysis, the background level for the minor allele was subtracted from the minor allele fraction to correct for the allele background. The allele background level can be estimated from pure DNA specimen and the difference between the reference and the alternative alleles is shown in FIGS. 8. As shown in FIGS. 8, the allele background was estimated from 48 samples, including replicates from 7 DNA specimens. 76 targets were observed as homozygous for the reference allele for at least one sample and 68 targets as homozygous for the alternative allele. For 47 targets, both homozygous reference allele and alternative allele were present among the 7 DNA specimens. The background level for reference and alternative alleles was plotted for the 47 targets. The background level for target was set to 10⁻⁵ if less or equal to 10⁻⁵. There were 22 targets with both reference and alternative alleles less or equal to 10⁻⁵, which are indicated by the circle. There were 30 targets with both reference and alternative alleles less or equal to 10⁻³ or 0.1%, which are indicated by the rectangle.

Digital Tag Sequences. Each capture probe contained a unique digital tag sequence. See FIG. 1B. After capturing, polymerization and ligation, a circular molecule was formed to include the target region with a unique molecular digital tag. After PCR and sequencing, the paired reads that contain the same digital tag sequence were grouped together as the PCR products of the same molecule and consensus sequence were derived as digital tag reads. The bias in PCR efficiency for each probe was corrected by counting the digital tag sequence reads for each allele at the target site.

Allele Frequency. At each target site, a MAF was estimated and the quality value for the consensus base-calls at this site is adjusted. Based on the MAF estimate, the posterior distribution of the genotypes was re-calculated. The constant of the proportionality was chosen so that the probabilities sum to 1. A genotype and quality value was assigned based on the maximum probability at each consensus position. Allele frequency improved log-likelihood ratio calculations by giving a more accurate estimate of the prior distribution, especially where the read coverage was low.

Weak Targets. Weak targets were excluded during the analysis. Weak targets consist of at least two types of probes: noisy probes and high CV probes. Noisy probe is defined by the poor performance in the pure DNA samples. In one exemplary embodiment, a noisy probe is a probe that has extremely low read count, for instance, less than 10 reads. In another exemplary embodiment, a noisy probe has a MAF between 0.05 to 0.35 when the genotype is homozygous for the target. The reason is that, for a pure sample, the probe MAF should be close to 0 if the target is homozygous or close to 0.5 if target is heterozygous. If the MAF deviates wildly from 0 or 0.5, it is categorized as a noisy probe.

The other type of weak probes is a high CV probe. A high CV probe is the probe that shows consistent high CV across 3 or more mixture samples in the training set where the MAF is greater than 0.25%. On the other hand, if one probe is showing very low CV across multiple samples and sequencing runs, it is a reliable probe which can be weighted higher in the model.

Background Level. Background level of a pure DNA sample is defined as the median of the MAF at all homozygous targets. For a validation set of pure DNA samples with repeats, the Limit of Blank (LoB) is defined as the 95^{th} percentile of the background levels of all samples, which under normal distribution is equivalent to LoB = mean_{blank} + 1.645(SD_{blank}). In this study, the LOB was calculated based on 48 samples including repeats from 7 pure DNA. To perform a fair comparison, background level and LOB were calculated the same way as in a previously published study. The previously published study showed LoB at 0.1% based on 180 blank samples which is marked with a solid line in FIG. 8B. In contrast, with the probes and methods provided herein, with a 192 probe set, the detection LoB was 0.0042%, marked by the dotted line, which is an order of magnitude lower than LoB by the methods available in the field.

Performance Characteristics. According to the Clinical and Laboratory Standards Institute EP17-A2, Limit of Detection (LOD) is defined as the minimum sample amount at which 95% of the samples are detectable. Here, LOD is defined as the lowest level of dd-cfDNA for which 95% of the samples including repeats are higher than the LOB. LoD is the lowest analyte concentration likely to be reliably distinguished from the LoB and at which detection is feasible. LoD is determined by utilizing both the measured LoB and test replicates of a sample known to contain a low concentration of analyte:

$$\text{LoD} = \text{LoB} + 1.645 \left(S {D}_{l o w m i x i n g l e v e l}\right)$$

Various mixing levels are tested with lowest level at 0.125%. Based on 16 samples with the lowest mixing level, LoD is 0.0478%.

### Example 5 - Determining the Donor Fraction in a Heterogeneous Sample

In this Example, a major and minor DNA were mixed to form a mixture sample, also called a heterogeneous sample. The major DNA represents the transplant recipient and the minor DNA represents the transplant donor. Mixtures of DNA from two DNA samples were prepared to simulate chimerism. The tested mixture levels were from 8% to 0.125%. Quantification accuracy in existing methods is poor and dependent on the quality of the DNA. The percentage of the minor DNA in these mixed samples represents approximately the targeted mixing level, i.e., the target donor fraction. In the first experiment, the target mixing levels were 1%, 0.5%, and 0.25%, each in triplicates, as shown in Table 20. Barcode is the plate and well location for each sample and must be unique for a particular mixing.

The actual mixing level (actual donor fraction) can be different from the target level because of pipetting errors. The actual mixing level is determined as the mean of the donor estimates of the 3 replicates. For each mixing level, Coefficient of Variation (CV) is calculated as the Standard Deviation divided by the Mean of donor estimates over the 3 replicates. The CV is represented as a percentage by multiplying 100. In this experiment, two pairs of DNA were used. For each pair, mixtures were made at mixing level 1%, 0.5% and 0.25%. Each mixture was triplicated. Two pure samples using the major DNA were included as the controls. In total, there were 22 sample in the sequencing run.

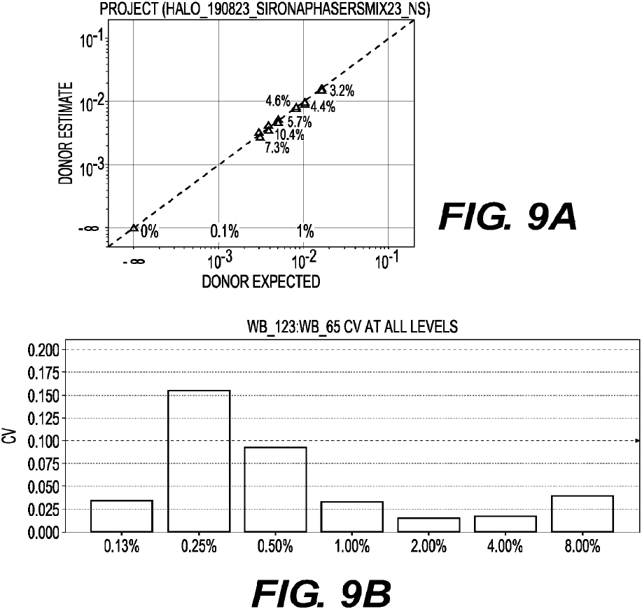

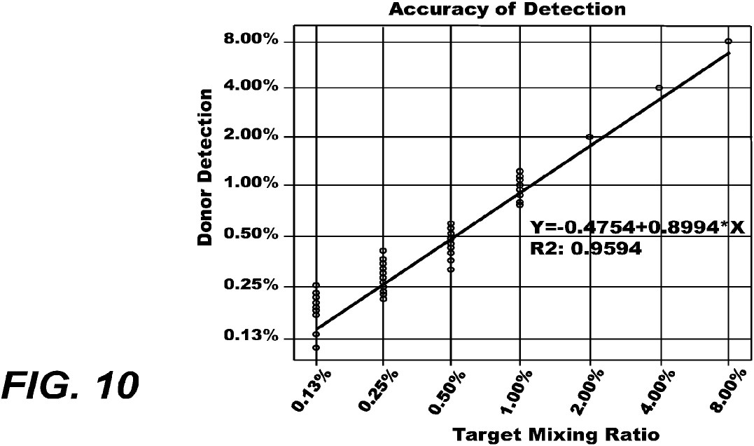

FIG. 9A shows an exemplary result of the correlation between the expected donor fraction in the mixture sample and the estimated donor fraction. The X axis represents the expected donor fraction which was calculated as the mean of the 3 replicates of the mixture sample at each mixing level. The Y axis shows the donor estimate for each replicate. Pure samples are shown at 0 for both donor expected and estimated.

TABLE 20

Sample level metrics for data analysis on the mixture experiment

| BARCODE | Reads | Target donor fraction | Number of informative markers | Actual donor fraction | CV |
|---|---|---|---|---|---|
| Plate04_A05 | 6187082 | 0.00% | 5 | 0.00% | |
| Plate04_A06 | 5521006 | 0.00% | 1 | 0.00% | |
| Plate04_B05 | 5775639 | 1.00% | 20 | 0.94% | 4.4% |
| Plate04_B06 | 5319402 | 0.91% | 18 | 0.94% | |
| Plate04_B07 | 3754514 | 0.92% | 21 | 0.94% | |
| Plate04_C05 | 7690315 | 0.54% | 19 | 0.50% | 5.7% |
| Plate04_C06 | 4853794 | 0.52% | 17 | 0.50% | |
| Plate04_C07 | 4883166 | 0.47% | 19 | 0.50% | |
| Plate04_D05 | 4250258 | 0.31% | 20 | 0.30% | 7.3% |
| Plate04_D06 | 3560967 | 0.32% | 20 | 0.30% | |
| Plate04_D07 | 4173906 | 0.27% | 17 | 0.30% | |
| Plate04_E05 | 4382701 | 0.00% | 2 | 0.00% | |
| Plate04_E06 | 3067983 | 0.00% | 11 | 0.00% | |
| Plate04_F05 | 4325737 | 1.65% | 27 | 1.58% | 3.2% |
| Plate04_F06 | 5195569 | 1.54% | 24 | 1.58% | |
| Plate04_F07 | 3673223 | 1.56% | 26 | 1.58% | |
| Plate04_G05 | 4596020 | 0.76% | 24 | 0.79% | 4.6% |
| Plate04_G06 | 5608809 | 0.84% | 27 | 0.79% | |
| Plate04_G07 | 4138259 | 0.76% | 25 | 0.79% | |
| Plate04_H05 | 5216784 | 0.43% | 25 | 0.38% | 10.4% |
| Plate04_H06 | 4467764 | 0.34% | 24 | 0.38% | |
| Plate04_H07 | 6662491 | 0.36% | 23 | 0.38% | |

In the second experiment, there were 7 mixing levels at 8%, 4%, 2%, 1%, 0.5%, 0.25%, and 0.125%. Each mixture sample had 3 replicates, as shown in Table 21. FIG. 9B shows that there is a negative correlation between the CV and the target donor fraction. Normally, the lower the donor fraction, the higher the CV of the estimates among the triplicate. Specifically, the lower the donor fraction, the closer the signal is to the noise level. When the signal-to-noise level is lower, the variation or CV is higher for detection. For all levels of donor fraction in the validation samples, the CV is below 20%.

TABLE 21

Target and actual mixing levels in the samples

| Major / Minor | Target donor fraction | Estimate in rep 1 | Estimate in rep 2 | Estimate in rep 3 | MEAN actual donor fraction | CV^{∗} |
|---|---|---|---|---|---|---|
| WB_123 / WB_65 | 0 (Pure) | 0.00% | 0.00% | 0.00% | 0.00% | NA |
| WB_123 / WB_65 | 8 | 7.84% | 8.12% | 7.36% | 7.77% | 4.03% |
| WB_123 / WB_65 | 4 | 4.01% | 4.14% | 4.16% | 4.10% | 1.67% |
| WB_123 / WB_65 | 2 | 1.90% | 1.95% | 1.97% | 1.94% | 1.46% |
| WB_123 / WB_65 | 1 | 0.96% | 1.00% | 0.92% | 0.96% | 3.29% |
| WB_123 / WB_65 | 0.5 | 0.55% | 0.51% | 0.44% | 0.50% | 9.31% |
| WB_123 / WB_65 | 0.25 | 0.30% | 0.39% | 0.27% | 0.32% | 15.54% |
| WB_123 / WB_65 | 0.125 | 0.19% | 0.21% | 0.20% | 0.20% | 3.38% |

What is claimed is:

1. A polynucleotide probe comprising two perfectly complementary strands, wherein one strand comprises, in a 5′ to 3′ direction,
a) a first target hybridization sequence,
b) a first digital tag sequence,
c) a first Halo barcode sequence,
d) a first Halo amplification primer sequence,
e) a reverse second Halo amplification primer sequence,
f) a reverse second Halo barcode sequence,
g) a reverse second digital tag sequence, and
h) a reverse second target hybridization sequence,
wherein the two strands are perfectly complementary to one another.

2. The polynucleotide probe of claim 1, further comprising a linker sequence between the first target hybridization sequence and the first digital tag sequence.

3. The polynucleotide probe of claim 2, further comprising a spacer sequence in between the first Halo amplification primer sequence and the reverse second Halo amplification primer sequence.

4. The polynucleotide probe of claim 3, wherein the spacer sequence is between 10-40 base pairs (bp) in length.

5. The polynucleotide probe of claim 4, wherein the spacer sequence is a non-human polynucleotide sequence.

6. The polynucleotide probe of claim 5, further comprising a linker sequence between the reverse second target hybridization sequence and the reverse second digital tag sequence.

7. The polynucleotide probe of any of claims 1-6, wherein the first target hybridization sequence and the reverse second target hybridization sequence are configured to hybridize to a single target polynucleotide sequence, wherein the target polynucleotide sequence is known to have more than one allele.

8. The polynucleotide probe of any one of claims 1-7, wherein the first target hybridization sequence and the reverse second target hybridization sequence are separated on the target polynucleotide sequence, when hybridized thereto, by a gap of at least 2 bp in length.

9. The polynucleotide probe of claim 8, wherein the gap is about 2 to about 1000 bp in length.

10. The polynucleotide probe of claim 8 or 9, wherein the gap is about 2 to about 800 bp in length.

11. The polynucleotide probe of any of claims 8-10, wherein the gap is about 2 to about 200 bp in length.

12. The polynucleotide probe of any of claims 1-11, wherein the polynucleotide is DNA.

13. A population of the polynucleotide probes of claim 12, wherein each member of the population of probes comprises the same first target hybridization sequence and the same reverse second target hybridization sequence.

14. A collection of polynucleotide probes, wherein the collection comprises more than one of the populations of probes of claim 13, wherein each population hybridizes to a different target polynucleotide sequence.

15. The collection of polynucleotide probes of claim 14, wherein at least two probes in the collection have the identical Halo barcode sequence and the identical reverse second Halo barcode sequence.

16. The collection of polynucleotide probes of claim 15, wherein all of the probes in the entire collection have the identical Halo barcode sequence and the identical reverse second Halo barcode sequence.

17. A method of amplifying a target polynucleotide sequence present in a sample, the method comprising:
a) denaturing the perfectly complementary strands of the polynucleotide probe of claim 12 to produce a first and second single stranded polynucleotide probe,
b) denaturing the target polynucleotide sequence present in the sample to produce a first and second single-stranded target polynucleotide sequences,
c) hybridizing each of the first and second single-stranded polynucleotide probes to the first and second single-stranded target polynucleotide sequences, respectively, wherein the single-stranded probes hybridize to the single-stranded target polynucleotide sequence in such a manner as to create circular hybrid polynucleotides, wherein the target hybridization sequences on the single-stranded polynucleotide probes are separated on the single-stranded target polynucleotide sequence, when hybridized thereto, by a gap of at least 2 nucleotides in length,
d) polymerizing with nucleotides in a 5′ to 3′ direction to fill in the gap of at least 2 nucleotides to produce a single-stranded circular probe, and
e) amplifying the single-stranded circular probe without cleaving the single-stranded circular probe, wherein amplification only occurs if the gap of at least 2 nucleotides is filled during the polymerization step.

18. The method of claim 17, wherein the target polynucleotide sequence is known to have more than one allele.

19. The method of claim 17 or 18, wherein amplifying the single-stranded circular probe comprises the use of at least four forward staggered amplification primers and four reverse staggered amplification primers.

20. The method of claim 19, wherein the at least four forward staggered amplification primers comprise the identical primer amplification polynucleotide sequence and the identical primer sequencing polynucleotide sequences, wherein the primer amplification polynucleotide sequence and the primer polynucleotide sequencing sequence are separated from one another by a spacer nucleotide sequence of 0, 1, 2 or 3 nucleotides in length, wherein the primer amplification polynucleotide sequence of the at least four forward staggered amplification primers are configured to hybridize to the first Halo amplification primer sequence of the single-stranded circular probe.

21. The method of claim 19, wherein the at least four reverse staggered amplification primers comprise the identical primer amplification polynucleotide sequence and the identical primer sequencing polynucleotide sequences, wherein the primer amplification polynucleotide sequence and the primer polynucleotide sequencing sequence are separated from one another by a spacer nucleotide sequence of 0, 1, 2 or 3 nucleotides in length, wherein the primer amplification polynucleotide sequence of the at least four reverse staggered amplification primers are configured to hybridize to the reverse second Halo amplification primer sequence of the single-stranded circular probe.

22. The method of any one of claims 17-21, wherein an exonuclease digestion is not performed at any time after the polymerization.

23. A method for determining a consensus sequence of at least one allele of a genetic variation of DNA in a sample obtained from a transplant recipient, wherein the sample contains at least recipient DNA, the method comprising:
(a) receiving a forward DNA sequencing read and a reverse DNA sequencing read, wherein each of the DNA sequencing reads comprises:
i). a first Halo barcode sequence and a second reverse Halo barcode sequence,
ii). a first digital tag sequence and a second reverse digital tag sequence,
iii). a target polynucleotide sequence, wherein the target polynucleotide sequence is known to be bi-allelic, and wherein the alleles are a non-single nucleotide polymorphism (SNP) genetic variation, and
iv). at least one index sequence;
(b) assigning the forward and reverse sequencing reads sharing the same index sequence to a single transplant recipient by mapping the index sequences to a reference index sequence, thereby producing one or more read clusters for the single transplant recipient, wherein each of the one or more read clusters comprise the forward and reverse target sequencing reads;
(c) verifying that the forward and reverse target sequencing reads are from the same sample preparation by confirming the sequence identity of the first and second reverse Halo barcode sequences;
(d) concatenating the first digital tag sequence and the second reverse digital tag sequence from each of the target sequencing reads in the read cluster to produce a long digital tag;
(e) identifying validated forward and reverse target sequencing reads in the read cluster by comparing the sequence of the long digital tag to a reference long digital tag sequence to confirm that there are no more than 2 mismatches between long digital tag and the reference long digital tag;
(f) aligning each of the validated forward and reverse target sequencing reads to target reference sequences, wherein the target reference sequences comprises one major allele of the non-SNP genetic variation or one minor allele of the non-SNP-genetic variation;
(g) generating a consensus sequence for the at least one allele for the target sequence for each of the one or more read clusters.

24. A method for determining a consensus sequence of at least one allele of a bi-allelic genetic variation of DNA in a sample obtained from a transplant recipient, wherein the sample contains at least recipient DNA, the method comprising:
(a) receiving a DNA sequencing read comprising:
i). a first Halo barcode sequence and a second reverse Halo barcode sequence,
ii). a first digital tag sequence and a second reverse digital tag sequence,
iii). a target polynucleotide sequence, wherein the target polynucleotide sequence is known to be bi-allelic, and wherein the alleles are a non-single nucleotide polymorphism (SNP) genetic variation, and
iv). at least one index sequence;
(b) assigning the sequencing reads sharing the same index sequence to a single transplant recipient by mapping the index sequences to a reference index sequence, thereby producing one or more read clusters for the single transplant recipient, wherein each of the one or more read clusters comprises the target sequencing read;
(c) verifying that the target sequencing reads are from the same sample preparation by confirming the sequence identity of the first and second reverse Halo barcode sequences;
(d) concatenating the first digital tag sequence and the second reverse digital tag sequence from each of the target sequencing reads in the read cluster to produce a long digital tag;
(e) identifying validated target sequencing reads in the read cluster by comparing the sequence of the long digital tag to a reference long digital tag sequence to confirm that there are no more than 2 mismatches between long digital tag and the reference long digital tag;
(f) aligning each of the validated target sequencing reads to target reference sequences, wherein each of the target reference sequences correspond to one allele of the bi-allelic genetic variation;
(g) generating a consensus sequence for the one allele of the bi-allelic genetic variation for each of the one or more read clusters.

25. The method of claim 23 or 24, wherein each of the DNA sequencing reads comprises a forward index sequence and a reverse index sequence.

26. The method of any one of claims 23-25, further comprising discarding low quality reads from the sequencing reads that fail a quality metrics.

27. The method of any one of claims 23-25, further comprising discarding a forward or reverse sequencing read if the index sequence comprises 2 or more mismatches compared to the reference index sequence.

28. The method of any one of claims 23-27, further comprising discarding that the forward and reverse target sequencing reads if the first and second reverse Halo barcode sequences comprise one or more mismatches to one another.

29. The method of any one of claims 23-28, further comprising discarding the validated forward target sequencing read and the validated reverse target sequencing read if they are not 100% complementary to each other.

30. The method of claim 23 or 24, wherein the consensus sequence for the target sequence for each read cluster is generated if the majority of the validated target sequencing reads align to the target reference sequences.

31. The method of any one of claims 23-30, further comprising storing the consensus sequence on a server.

32. The method of any one of claims 23-31, wherein the DNA is cell-free DNA.

33. The method of any one of claims 23-32, wherein the sample comprises blood, serum, plasma, peripheral blood mononuclear cells (PBMCs), cells, tissues, biopsies, cerebrospinal fluid, bile, lymph fluid, saliva, urine, and stool.

34. The method of any one of claims 23-33, wherein the non-SNP genetic variation is selected from the group consisting of insertions, deletions, variable number of tandem repeats (VNTRs), duplication, repeats, hypervariable regions, minisatellites, copy number variation, translocation, and inversion.

35. The method of any one of claims 23-34, wherein the minor allele of the non-SNP genetic variation is known to have an occurrence in a population of no lower than about 30%.

36. The method of any one of claims 23-35, wherein the first digital tag sequence or the second reverse digital tag sequence comprises between 8 to 20 nucleotides.

37. The method of claim 36, wherein the forward first digital tag sequence or the second reverse digital tag sequence comprises 12 nucleotides.

38. The method of any one of claims 23-37, wherein the sample contains a mixture of donor DNA and recipient DNA, and wherein the donor and the recipient are unrelated.

39. A computer-readable storage medium comprising instructions stored thereon, when executed in a computerized system comprising at least one processor, to cause the at least one processor to carry out the method of any one of claims 23-38.

40. A method of determining a donor fraction of cell-free DNA in a sample obtained from a transplant recipient comprising at least recipient cell-free DNA, the method comprising:
a) identifying a subset of informative markers, selected from a pre-determined master set of genetic variations, wherein each of the genetic variations within the master set of genetic variations are known to be bi-allelic and wherein the allele in the bi-allelic pair is a non-single nucleotide polymorphism (SNP) genetic variation, wherein the identification of the subset of informative markers comprises,
i) determining the polynucleotide sequence of all of a target set of polynucleotide sequences in the sample, wherein the target sequences correspond to the master set of genetic variations,
ii) determining a sample minor allele frequency of each of the master set of genetic variations within the sample, and
iii) identifying the subset of informative markers based on the sample minor allele frequency in the sample being equal to or greater than 0.05%,
b) estimating an initial probability of observing the genotype of each of the informative markers in the sample, based on an accepted frequency of each allele of the informative markers across a population of individuals,
c) calculating an initial donor faction estimate of cell-free DNA from the estimated initial probabilities of observing the frequency of the sample minor alleles,
d) calculating a conditional probability of observing the frequency of the sample minor allele from the calculated initial donor faction estimate and the standard deviation of an observed frequency of the sample minor alleles,
e) applying a mixture model algorithm to the calculated initial donor faction estimate to provide an updated donor faction estimate of cell-free DNA in the sample, wherein steps (c)-(d) are repeated using the updated donor fraction of cell-free DNA in place of the initial donor faction estimate of cell-free DNA until the absolute value of the change in the updated donor faction estimate is less than a pre-set threshold value.

41. The method of claim 40, wherein the pre-set threshold value is 1.0E-6 or lower.

42. The method of claim 40 or 41, wherein the pre-set threshold value is in the range of 1.0E-12 to 1.0E-6, inclusive.

43. The method of any one of claims 40-42, wherein the sample minor allele frequency in the sample is less than about 20%.

44. The method of any one of claims 40-43, further comprising identifying the sample as not comprising donor fraction of cell-free DNA if the subset of informative markers comprises less than or equal to 3 informative markers.

45. The method of any one of claims 40-44, wherein the accepted frequency of each allele of the informative markers is known to have an occurrence in a population of no lower than about 30%.

46. The method of any one of claims 40-45, wherein the conditional probability of observing the frequency of the sample minor allele in the sample is calculated from the mean of a probability distribution chosen from an exponential family of the estimated initial probabilities of observing the frequency of the sample minor alleles.

47. The method of claim 46, wherein the form of probability distribution is selected from the group consisting of two parameter Gaussian distribution, two parameter Gamma distribution, and multinomial distribution.

48. The method of any one of claims 40-47, wherein the transplant recipient is homozygous for each of the informative markers.
